(19) United States
(12) Patent Application Publication
(10) Pub. No.: US 2023/0099898 A1
(43) Pub. Date: Mar. 30, 2023

(54) COMPOSITE RNA PARTICLES

(71) Applicants: BioNTech SE, Mainz (DE); Johannes Gutenberg-Universitat Mainz, Mainz (DE)

(72) Inventors: Heinrich HAAS, Mainz (DE); Sara NOGUEIRA, Mainz (DE); Peter LANGGUTH, Biebergemünd (DE); Christian SIEWERT, Weimar (DE); Vera CORNET, Mainz (DE)

(21) Appl. No.: 17/904,937

(22) PCT Filed: Feb. 26, 2021
(86) PCT No.: PCT/EP2021/054843

§ 371 Date: Aug. 24, 2022

(30) Foreign Application Priority Data
Mar. 2, 2020 (WO) PCT/EP2020/055410

(51) Int. Cl. A61K 9/16; C12N 15/88; A61K 31/7105

(52) U.S. Cl. CPC A61K 9/1617; A61K 9/1658; A61K 9/1694; A61K 31/7105; C12N 15/88; A61K 48/00

(57) ABSTRACT

The present disclosure relates to RNA particles for delivery of RNA to target tissues after administration, in particular after parenteral administration such as intramuscular, intravenous, subcutaneous or intratumoral administration, and compositions comprising such RNA particles. The present disclosure, in particular, relates to RNA particles comprising RNA, at least one cationic or cationically ionizable lipid or lipid-like material, and at least one cationic polymer, wherein the particles do not have a core-shell structure.

### TECHNICAL FIELD

The present disclosure relates to RNA particles for delivery of RNA to target tissues after administration, in particular after parenteral administration such as intramuscular, intravenous, subcutaneous or intratumoral administration, and compositions comprising such RNA particles. The present disclosure, in particular, relates to RNA particles comprising RNA, at least one cationic or cationically ionizable lipid or lipid-like material, and at least one cationic polymer, wherein the particles do not have a core-shell structure. In one embodiment, the RNA particles have a composite structure wherein the at least one cationic or cationically ionizable lipid or lipid-like material and the at least one cationic polymer are not present as two separate layers but rather in a mixed structure. The RNA particles in one embodiment comprise single-stranded RNA such as mRNA which encodes a peptide or protein of interest, such as a pharmaceutically active peptide or protein. The RNA is taken up by cells of a target tissue and the RNA is translated into the encoded peptide or protein, which may exhibit its physiological activity. The peptide or protein of interest may be a peptide or protein comprising one or more epitopes for inducing or enhancing an immune response directed against the one or more epitopes.

### BACKGROUND

Messenger RNA (mRNA)-based drugs are a new class of therapeutics for versatile medical applications. mRNA can encode key information such as vaccine peptides. The use of RNA for delivery of foreign genetic information into target cells offers an attractive alternative to DNA. The advantages of using RNA include transient expression and a non-transforming character. RNA does not need to enter the nucleus in order to be expressed and moreover cannot integrate into the host genome, thereby eliminating the risk of oncogenesis. Problems associated with the use of mRNA are due to instability, rapid degradation via ribonuclease (RNase) enzymes and low cellular uptake. Suitable formulations or vehicles are required to allow the use of RNA as an active pharmaceutical component in pharmaceutical products for patients.

RNA may be delivered to a subject using different delivery vehicles, mostly based on cationic polymers or lipids which together with the RNA form nanoparticles. The nanoparticles are intended to protect the RNA from degradation, enable delivery of the RNA to the target site and facilitate cellular uptake and processing by the target cells. One approach is based on the combination of polycationic polymers with positively charged lipids that complex the mRNA via electrostatic interactions.

Various types of nanoparticles of lipids and polymers are known. Hybrid particles of polymers and lipids have also been described. The combination of protamine and DOTAP has previously been used e.g. in systemic gene therapy of the E1A gene and herpes simplex virus 1-thymidine kinase (Ueno, N.T. et al., Cancer Res. 62 (2002) 6712-6716; Wang, K. et al., Pharmacol. Res. 114 (2016) 56-66; Wang, Y. et al., Mol. Ther. 21 (2013) 358-367). The effect of the combination of both is beneficial for the transport of DNA (Nchinda, G. et al., BMC Biotechnol. 2 (2002) 12; Arangoa, M.A. et al., Gene Ther. 10 (2003) 5-14; Li, S. et al., Gene Ther. 5 (1998) 930-937; Li, S. et al., Gene Ther. 4 (1997) 891-900; Sorgi, F. et al., Gene Ther. 4 (1997) 961-968; Pozzi, D. et al., Mol. Pharm. 10 (2013) 4654-4665).

However, there is a need for improving previously existing nanoparticle formulations for the application of RNA in pharmaceutical products.

Here, RNA preparations are described comprising a cationic polymer as well as a cationic lipid. RNA particles obtained from two different approaches for preparing particles having a core-shell structure were compared to RNA particles obtained from a one-step manufacturing protocol for composite particle formation. In the latter case, a mixture of cationic polymer and cationic lipid is first prepared, with which then the RNA is complexed. The relationship between the preparation, structure, and effectiveness of the nanoparticulate systems has been studied in detail. An unexpected difference in the effectiveness of the particles to deliver RNA resulting from differences in the sequence of the assembly of the systems and the resulting altered particle structure was observed with qualitative and quantitative similar compositions of the systems.

Hybrid systems of cationic lipids and polymers with a core-shell organization have been described in the past as nucleic acid delivery systems. Here, mixed systems are described in which the two cationic components are mixed to form a composite-like structure. It is demonstrated that such mixed systems have improved efficiency over systems which are sequentially assembled to obtain a core-shell organization when used as RNA transfection systems. In particular, a correlation between the molecular organization resulting from different manufacturing protocols and the functional transfection capacity of the particles in vitro and in vivo was observed. The combination of the two complexing agents, cationic lipid and cationic polymer, in a mixed system resulted in improved transfection of mRNA compared to systems wherein the individual complexing agents are sequentially adsorbed and present in separate layers. A single-step protocol wherein the complexing agents are mixed together with appropriate protocols before mRNA addition results in composite nanoparticles with a characteristic internal organization which results in higher transfection efficiency compared to core-shell structures derived from previously reported sequential assembly processes. Based on these observations improved pharmaceutical RNA products can be developed.

### SUMMARY

In one aspect, the present disclosure relates to an RNA particle comprising:
(i) RNA,
(ii) at least one cationic or cationically ionizable lipid or lipid-like material, and
(iii) at least one cationic polymer,
wherein the particle does not have a core-shell structure.

In one embodiment, the RNA particle is obtainable by a process comprising the following steps:
a. obtaining a mixture comprising at least one cationic or cationically ionizable lipid or lipid-like material and at least one cationic polymer;
b. obtaining a colloid from the mixture obtained in step a.; and
c. mixing the colloid obtained in step b. with RNA to obtain RNA particles.

In one embodiment, the cationic or cationically ionizable lipid or lipid-like material prior to mixing with the cationic polymer is present in a non-aqueous phase, e.g., in an organic solvent, and/or the cationic polymer prior to mixing with the cationic or cationically ionizable lipid or lipid-like material is present in an aqueous phase.

In one embodiment, the process comprises removing the non-aqueous solvent from the mixture obtained in step a.

In one embodiment, the cationic or cationically ionizable lipid or lipid-like material prior to mixing with the cationic polymer is present in dry form, and/or the cationic polymer prior to mixing with the cationic or cationically ionizable lipid or lipid-like material is present in an aqueous phase.

In one embodiment, the RNA particle is obtainable by a process comprising the following steps:
a. obtaining a mixture comprising at least one cationic or cationically ionizable lipid or lipid-like material in an organic solvent;
b. removing the organic solvent from the mixture obtained in step a. thus obtaining a lipid film;
c. adding a solution, e.g., an aqueous solution, comprising at least one cationic polymer to the lipid film obtained in step b.;
d. agitating the mixture obtained in step c. to obtain a colloid; and
e. mixing the colloid obtained in step d. with RNA to obtain RNA particles.

In one aspect, the present disclosure relates to an RNA particle comprising:
(i) RNA,
(ii) at least one cationic or cationically ionizable lipid or lipid-like material, and
(iii) at least one cationic polymer,

wherein the RNA particle is obtainable by a process comprising the following steps:
a. obtaining a mixture comprising at least one cationic or cationically ionizable lipid or lipid-like material and at least one cationic polymer;
b. obtaining a colloid from the mixture obtained in step a.; and
c. mixing the colloid obtained in step b. with RNA to obtain RNA particles.

In one embodiment, the cationic or cationically ionizable lipid or lipid-like material prior to mixing with the cationic polymer is present in a non-aqueous phase, e.g., in an organic solvent, and/or the cationic polymer prior to mixing with the cationic or cationically ionizable lipid or lipid-like material is present in an aqueous phase.

In one embodiment, the process comprises removing the non-aqueous solvent from the mixture obtained in step a.

In one embodiment, the cationic or cationically ionizable lipid or lipid-like material prior to mixing with the cationic polymer is present in dry form, and/or the cationic polymer prior to mixing with the cationic or cationically ionizable lipid or lipid-like material is present in an aqueous phase.

In one aspect, the present disclosure relates to an RNA particle comprising:
(i) RNA,
(ii) at least one cationic or cationically ionizable lipid or lipid-like material, and
(iii) at least one cationic polymer,

wherein the RNA particle is obtainable by a process comprising the following steps:
a. obtaining a mixture comprising at least one cationic or cationically ionizable lipid or lipid-like material in an organic solvent;
b. removing the organic solvent from the mixture obtained in step a. thus obtaining a lipid film;
c. adding a solution, e.g., an aqueous solution, comprising at least one cationic polymer to the lipid film obtained in step b.;
d. agitating the mixture obtained in step c. to obtain a colloid; and
e. mixing the colloid obtained in step d. with RNA to obtain RNA particles.

In one embodiment, step c. (adding a solution comprising at least one cationic polymer to the lipid film obtained in step b.) results in hydration of the lipid film.

In one embodiment, the process described herein, in particular step d. (agitating the mixture obtained in step c. to obtain a colloid) comprises reducing the size of the particles of the colloid obtained.

In one embodiment, the process described herein, in particular step d. (agitating the mixture obtained in step c. to obtain a colloid) comprises applying sonic energy or mechanical energy to obtain a colloid.

In one embodiment, the process described herein, in particular step d. (agitating the mixture obtained in step c. to obtain a colloid) comprises one or more selected from the group consisting of sonification, extrusion, dual asymmetric centrifugation, vortexing and compounding to obtain a colloid.

In one embodiment, the process described herein, in particular step d. (agitating the mixture obtained in step c. to obtain a colloid) comprises dual asymmetric centrifugation to obtain a colloid.

In one embodiment, the process described herein does not comprise preparing complexes by mixing RNA and cationic polymer in the absence of cationic or cationically ionizable lipid or lipid-like material.

In one embodiment, the process described herein does not comprise preparing a colloid comprising cationic or cationically ionizable lipid or lipid-like material in the absence of cationic polymer.

In one embodiment, the process described herein does not comprise mixing RNA with cationic polymer in the absence of cationic or cationically ionizable lipid or lipid-like material.

In one embodiment, the process described herein does not comprise mixing RNA with cationic or cationically ionizable lipid or lipid-like material in the absence of cationic polymer.

In one embodiment of all aspects described herein, one or more, preferably all of the components (i), (ii) and (iii) are distributed throughout the particle.

In one embodiment of all aspects described herein, the RNA particle does not have a core-shell structure. In one embodiment of all aspects described herein, the RNA particle does not consist of a particle core comprising RNA and an outer shell. In one embodiment of all aspects described herein, the RNA particle does not consist of a particle core and an outer shell, wherein the particle core comprises RNA and cationic polymer and the outer shell is a lipid shell comprising cationic or cationically ionizable lipid or lipid-like material. In one embodiment of all aspects described herein, the RNA particle does not consist of a particle core and an outer shell, wherein the particle core comprises RNA and cationic or cationically ionizable lipid or lipid-like material and the outer shell comprises cationic polymer.

In one embodiment of all aspects described herein, the cationically ionizable lipid or lipid-like material is cationic only at acidic pH and does not remain cationic at neutral pH.

In one embodiment of all aspects described herein, the cationic or cationically ionizable lipid or lipid-like material comprises N-(1-(2,3-dioleoyloxy)propyl)-N,N,N-trimethylammonium chloride (DOTAP), N,N-dimethyl-2,3-dioleyloxypropylamine (DODMA), N,N-dioleyl-N,N-dimethylammonium chloride (DODAC), N,N-distearyl-N,N-dimethylammonium bromide (DDAB), N-(1-(2,3-dioleyloxy)propyl)-N,N,N-trimethylammonium chloride (DOTMA), 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-KC2-DMA), 2,2-dilinoleyl-4-dimethylaminomethyl-[1,3]-dioxolane (DLin-K-DMA), 1,2-dimyristyloxypropyl-3-dimethyl-hydroxy ethyl ammonium bromide (DMRIE), (6Z,9Z,28Z,31Z)-heptatriaconta-6,9,28,31-tetraen-19-yl 4-(dimethylamino)butanoate (D-Lin-MC3-DMA) or a mixture thereof.

In one embodiment of all aspects described herein, the cationic or cationically ionizable lipid or lipid-like material comprises N-(1-(2,3-dioleoyloxy)propyl)-N,N,N-trimethylammonium chloride (DOTAP).

In one embodiment of all aspects described herein, the particles described herein further comprise a non-cationic lipid or lipid-like material. In one embodiment, the non-cationic lipid or lipid-like material comprises a phospholipid. In one embodiment, the non-cationic lipid or lipid-like material comprises cholesterol or a cholesterol derivative. In one embodiment, the non-cationic lipid or lipid-like material comprises a mixture of a phospholipid and cholesterol or a cholesterol derivative. In one embodiment, the phospholipid is selected from the group consisting of distearoylphosphatidylethanolamine (DSPE), distearoylphosphatidylcholine (DSPC), dipalmitoylphosphatidylcholine (DPPC), or a mixture thereof. In one embodiment, the non-cationic lipid or lipid-like material comprises a mixture of DSPE and cholesterol.

In one embodiment of all aspects described herein, the cationic lipid or lipid-like material comprises from about 20 mol % to about 100 mol % of the total lipid and lipid-like material present in the particles (particles contained in the colloid described herein and RNA particles described herein).

In one embodiment of all aspects described herein, the non-cationic lipid or lipid-like material comprises from about 0 mol % to about 80 mol % of the total lipid and lipid-like material present in the particles (particles contained in the colloid described herein and RNA particles described herein).

In one embodiment of all aspects described herein, the particles described herein do not comprise a polyethyleneglycol-lipid conjugate or a conjugate of polyethyleneglycol and a lipid-like material, and preferably do not comprise polyethyleneglycol. However, in some embodiments, the particles described herein may comprise a polyethyleneglycol-lipid conjugate or a conjugate of polyethyleneglycol and a lipid-like material, or may comprise polyethyleneglycol

In one embodiment of all aspects described herein, the cationic polymer is a nucleic acid condensing agent. In one embodiment of all aspects described herein, the cationic polymer is a cationic peptide or protein. In one embodiment of all aspects described herein, the cationic polymer comprises one or more selected from the group consisting of protamine, spermidine (N-[3-aminopropyl]-1,4-butanediamine), DEAE-dextran, chitosan, polyethyleneimine and poly-lysine. In one embodiment of all aspects described herein, the cationic polymer comprises protamine.

In one embodiment of all aspects described herein, the RNA is mRNA or saRNA.

In one embodiment of all aspects described herein, the particle is a nanoparticle.

In one embodiment of all aspects described herein, the particle has a size of from about 100 nm to about 300 nm.

In one embodiment of all aspects described herein, the particle is a non-viral particle.

In one aspect, the present disclosure relates to composition comprising a plurality of the particles described herein.

In one aspect, the present disclosure relates to a method for delivering RNA to cells of a subject, the method comprising administering to a subject a plurality of the particles described herein or the composition described herein.

In one aspect, the present disclosure relates to a method for delivering a therapeutic peptide or protein to a subject, the method comprising administering to a subject a plurality of the particles described herein or the composition described herein, wherein the RNA encodes the therapeutic peptide or protein.

In one aspect, the present disclosure relates to a method for treating or preventing a disease or disorder in a subject, the method comprising administering to a subject a plurality of the particles described herein or the composition described herein, wherein delivering the RNA to cells of the subject is beneficial in treating or preventing the disease or disorder.

In one aspect, the present disclosure relates to a method for treating or preventing a disease or disorder in a subject, the method comprising administering to a subject a plurality of the particles described herein or the composition described herein, wherein the RNA encodes a therapeutic peptide or protein and wherein delivering the therapeutic peptide or protein to the subject is beneficial in treating or preventing the disease or disorder.

In one aspect, the present disclosure relates to particles described herein or a composition described herein for use in a method described above.

In one embodiment of all aspects described herein, the subject is a mammal. In one embodiment of all aspects described herein, the mammal is a human.

In one aspect, the present disclosure relates to a process for the preparation of RNA particles comprising the following steps:
a. obtaining a mixture comprising at least one cationic or cationically ionizable lipid or lipid-like material and at least one cationic polymer;
b. obtaining a colloid from the mixture obtained in step a.; and
c. mixing the colloid obtained in step b. with RNA to obtain RNA particles.

In one embodiment, the cationic or cationically ionizable lipid or lipid-like material prior to mixing with the cationic polymer is present in a non-aqueous phase, e.g., in an organic solvent, and/or the cationic polymer prior to mixing with the cationic or cationically ionizable lipid or lipid-like material is present in an aqueous phase.

In one embodiment, the process comprises removing the non-aqueous solvent from the mixture obtained in step a.

In one embodiment, the cationic or cationically ionizable lipid or lipid-like material prior to mixing with the cationic polymer is present in dry form, and/or the cationic polymer prior to mixing with the cationic or cationically ionizable lipid or lipid-like material is present in an aqueous phase.

In one aspect, the present disclosure relates to a process for the preparation of RNA particles comprising the following steps:
a. obtaining a mixture comprising at least one cationic or cationically ionizable lipid or lipid-like material in an organic solvent;
b. removing the organic solvent from the mixture obtained in step a. thus obtaining a lipid film;
c. adding a solution, e.g., an aqueous solution, comprising at least one cationic polymer to the lipid film obtained in step b.;
d. agitating the mixture obtained in step c. to obtain a colloid; and
e. mixing the colloid obtained in step d. with RNA to obtain RNA particles.

In one embodiment, step c. (adding a solution comprising at least one cationic polymer to the lipid film obtained in step b.) results in hydration of the lipid film.

In one embodiment, the process described herein, in particular step d. (agitating the mixture obtained in step c. to obtain a colloid) comprises reducing the size of the particles of the colloid obtained.

In one embodiment, the process described herein, in particular step d. (agitating the mixture obtained in step c. to obtain a colloid) comprises applying sonic energy or mechanical energy to obtain a colloid.

In one embodiment, the process described herein, in particular step d. (agitating the mixture obtained in step c. to obtain a colloid) comprises one or more selected from the group consisting of sonification, extrusion, dual asymmetric centrifugation, vortexing and compounding to obtain a colloid.

In one embodiment, the process described herein, in particular step d. (agitating the mixture obtained in step c. to obtain a colloid) comprises dual asymmetric centrifugation to obtain a colloid.

In one embodiment, the process described herein does not comprise preparing complexes by mixing RNA and cationic polymer in the absence of cationic or cationically ionizable lipid or lipid-like material.

In one embodiment, the process described herein does not comprise preparing a colloid comprising cationic or cationically ionizable lipid or lipid-like material in the absence of cationic polymer.

In one embodiment, the process described herein does not comprise mixing RNA with cationic polymer in the absence of cationic or cationically ionizable lipid or lipid-like material.

In one embodiment, the process described herein does not comprise mixing RNA with cationic or cationically ionizable lipid or lipid-like material in the absence of cationic polymer.

Further embodiments of the process aspects described herein are as described above for the RNA particles. In one embodiment of all process aspects described herein, the particles, in particular RNA particles are particles as described herein.

In further aspects, the present disclosure relates to a colloid described herein (e.g., prepared by process steps for preparing a colloid as described herein) and a process for preparing a colloid comprising process steps for preparing a colloid as described herein.

In one embodiment of all aspects described herein, the colloid is a colloid of mixed cationic or cationically ionizable lipid or lipid-like material and cationic polymer. In one embodiment of all aspects disclosed herein, the RNA particle comprises a mixture of cationic or cationically ionizable lipid or lipid-like material and cationic polymer.

### BRIEF DESCRIPTION OF THE DRAWINGS

FIG. 1: Classical core-shell particles and composite particles. Shown are three different structural morphologies obtained by variation of the assembly routes.

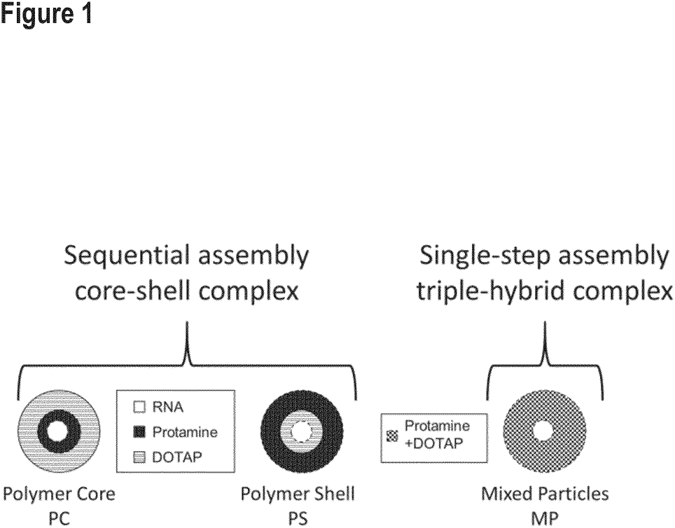

FIG. 2: Particle assembly strategies. Shown are different assembly routes for preparation of different particle topologies with protamine as either the core (I, PC), the shell (II, PS), or self-assembled/mixed particle (III, MP) component.

FIG. 3: Preparation of hybrid lipid/polymer particles. Hybrid lipid/polymer particles were prepared while varying the manufacturing method, as well as the composition. The nomenclature is given by the three different assembly routes (PC, PS, MP) and the amount of protamine contribution to the positive charge (low, middle and high amount of protamine charge contribution, written as _{Low,} _{MID} _{and} _{HIGH}). The black colored columns represent the core composition of the respective assembly route. The sum of the N/P ratio is equal 2 for all particles. The weight ratio is given in percent protamine:DOTAP:RNA (P:D:R).

FIG. 4: Physicochemical characterization of the different types of hybrid particles. Physicochemical characterization of the three different particle topologies, each with three different protamine contributions to the positive charge. The mean particle size, given as Z_{average} (Z_{Ave}, second column), the polydispersity index (PDI, third column) and the zeta potential (ZP, fourth column) were obtained from DLS/ELS and accessible mRNA (mRNA, right column) was measured using the Quant-iT™ RiboGreen® kit. All particles display suitable properties as required for parenteral application of pharmaceutical products. In particular, the particle size is below 500 nm and the polydispersity index is below 0.5.

FIG. 5: Release of mRNA from the particles by applying an excess of heparin. Three different particle topologies, as well as DOTAP/mRNA and Protamine/mRNA particles were tested. The measurement was performed to investigate how easily the RNA is released from the particles in order to mimic endosomal release. The RNA in the composite particles is released more easily than the one in the particles with core-shell organization.

FIG. 6: In vitro test of the different types of polymer/lipid formulations. Transfection of HEK293 cells was tested for the three formulations. The activity of the particles with composite organization was at least one order of magnitude higher than that of the particles with an organization obtained from the core-shell protocol.

FIG. 7: In vivo evaluation of the particles with the different architectures. Top: Bioluminescence imaging of mice (n=3 for particles, n=2 for negative control) 6, 24 and 48 hours after i.m. injection in the tight of either PC_{MID}, PS_{MID}, MP_{MID}, Protamine/mRNA, DOTAP/mRNA or only PBS buffer. Bottom left: Total flux of the samples with respect to the time. Bottom right: Area under the curve (AUC) of the respective samples. Statistical analysis was performed using one-way ANOVA with Tukey’s multiple comparisons test.

FIG. 8: Drug carrier structure estimation by SANS and cryo-TEM. (A) Double logarithmic plot of SANS scattering profiles of particles in HBG buffer (pH 7.2). Scattering curves of PC_{LOW} and PC_{LOW} intermediate, PS_{HIGH} and PS_{HIGH} intermediate and PS_{MID} at 77% D₂O. The curves are vertically shifted for clarity. The scattering curves follow a linear slope determined for the q-range 0.005-0.02 Å⁻¹. (B) Intensity scattering of PC_{LOW} particles at q-range of 0.03-0.18 Å⁻¹ at different D₂O concentrations. The peak at 0.1 Å⁻¹ is present when the scattering length density of the buffer diverges from the scattering length density of the lipid. (C, D) cryo-TEM images of PS_{HIGH} (C) and PC_{LOW} (D).

FIG. 9: Drug carrier structure estimation by SAXS and cryo-TEM. (A) Double logarithmic plot of SAXS scattering profiles of particles in HBG buffer (pH 7.2). Scattering curves of MP_{MID} intermediate and MP_{MID}. The curves are vertically shifted for clarity. A small peak is present in the scattering curve of MP_{MID} but not for the intermediate at 0.1 Å⁻¹ (B) Cryo-TEM image of MP_{MID}.

FIG. 10: Analysis by small angle x-ray scattering. Left: Double logarithmic plot of SAXS scattering data of particles with an N/P ratio of 2 in HBGx1 buffer (pH 7.2). Curves are shifted vertically for clarity. Comparison of pure DOTAP, pure Protamine, PC_{MID}, PS_{MID}, MP_{MID} intermediate and MP_{MID}. The scattering curves follow a linear slope, highlighted by the straight lines next to the curves for the q-range 0.005-0.02 Å⁻¹. Right: Kratky plot (bottom left inset plot) was obtained by plotting I(q)*q2 against q. For better comparison of the curves, the intensity is shifted vertically and adjusted to compare the peak maxima at 0.075 Å⁻¹.

### DETAILED DESCRIPTION

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H.G.W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present disclosure will be described. These elements are listed with specific embodiments, however, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise.

The term “about” means approximately or nearly, and in the context of a numerical value or range set forth herein in one embodiment means ± 20%, ± 10%, ± 5%, or ± 3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e. for the purpose of this embodiment “comprising” is to be understood as having the meaning of “consisting of”.

Several documents are cited throughout the text of this specification. Each of the documents cited herein (including all patents, patent applications, scientific publications, manufacturer’s specifications, instructions, etc.), whether supra or infra, are hereby incorporated by reference in their entirety. Nothing herein is to be construed as an admission that the present disclosure was not entitled to antedate such disclosure.

### Definitions

In the following, definitions will be provided which apply to all aspects of the present disclosure. The following terms have the following meanings unless otherwise indicated. Any undefined terms have their art recognized meanings.

Terms such as “reduce” or “inhibit” as used herein means the ability to cause an overall decrease, for example, of about 5% or greater, about 10% or greater, about 20% or greater, about 50% or greater, or about 75% or greater, in the level. The term “inhibit” or similar phrases includes a complete or essentially complete inhibition, i.e. a reduction to zero or essentially to zero.

Terms such as “increase” or “enhance” in one embodiment relate to an increase or enhancement by at least about 10%, at least about 20%, at least about 30%, at least about 40%, at least about 50%, at least about 80%, or at least about 100%.

“Physiological pH” as used herein refers to a pH of about 7.4.

As used in the present disclosure, “% w/v” refers to weight by volume percent, which is a unit of concentration measuring the amount of solute in grams (g) expressed as a percent of the total volume of solution in milliliters (mL).

As used in the present disclosure, “mol %” is defined as the ratio of the number of moles of one component to the total number of moles of all components, multiplied by 100.

The term “ionic strength” refers to the mathematical relationship between the number of different kinds of ionic species in a particular solution and their respective charges. Thus, ionic strength I is represented mathematically by the formula

$$I = \text{} \frac{1}{2} ⋅ {\sum }_{i} {z}_{i}^{2} ⋅ {c}_{i}$$

in which c is the molar concentration of a particular ionic species and z the absolute value of its charge. The sum Σ is taken over all the different kinds of ions (i) in solution.

According to the disclosure, the term “ionic strength” in one embodiment relates to the presence of monovalent ions. Regarding the presence of divalent ions, in particular divalent cations, their concentration or effective concentration (presence of free ions) due to the presence of chelating agents is in one embodiment sufficiently low so as to prevent degradation of the RNA. In one embodiment, the concentration or effective concentration of divalent ions is below the catalytic level for hydrolysis of the phosphodiester bonds between RNA nucleotides. In one embodiment, the concentration of free divalent ions is 20 µM or less. In one embodiment, there are no or essentially no free divalent ions.

“Osmolality” refers to the concentration of solutes expressed as the number of osmoles of solute per kilogram of solvent.

The term “freezing” relates to the phase transition from the liquid to the solid state. It usually occurs on lowering the temperature of a system below a critical temperature and is accompanied by a characteristic change of enthalpy of the system.

The term “lyophilizing” or “lyophilization” refers to the freeze-drying of a substance by freezing it and then reducing the surrounding pressure to allow the frozen medium in the substance to sublimate directly from the solid phase to the gas phase.

The term “spray-drying” refers to spray-drying a substance by mixing (heated) gas with a fluid that is atomized (sprayed) within a vessel (spray dryer), where the solvent from the formed droplets evaporates, leading to a dry powder.

The term “cryoprotectant” relates to a substance that is added to a formulation in order to protect the active ingredients during the freezing stages.

The term “lyoprotectant” relates to a substance that is added to a formulation in order to protect the active ingredients during the drying stages.

The term “reconstitute” relates to adding a solvent such as water to a dried product to return it to a liquid state such as its original liquid state.

The term “recombinant” in the context of the present disclosure means “made through genetic engineering”. In one embodiment, a “recombinant object” in the context of the present disclosure is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

In the context of the present disclosure, the term “particle” relates to a structured entity formed by molecules or molecule complexes. In one embodiment, the term “particle” relates to a micro-or nano-sized structure, such as a micro- or nano-sized compact structure dispersed in a medium.

In the context of the present disclosure, the term “RNA particle” relates to a particle that contains RNA. Electrostatic interactions between positively charged molecules such as polymers and lipids and negatively charged RNA are involved in particle formation. This results in complexation and spontaneous formation of RNA particles. In one embodiment, a RNA particle is a nanoparticle.

As used in the present disclosure, “nanoparticle” refers to a particle having an average diameter suitable for parenteral administration.

In the context of the present disclosure, the term “core-shell structure”, “core-shell organization” or a similar term as used herein in reference to RNA particles relates to particles which are sequentially assembled. In one embodiment, a core material is first added to RNA, which is followed by adding shell material. This is in contrast to the production of particles (particles having a composite structure or mixed structure), wherein core material and shell material are mixed first and then RNA is added. In one embodiment, the term “core-shell structure”, “core-shell organization” or a similar term refers to particles comprising an inner core that is either partially or wholly covered or otherwise surrounded by an outer shell layer. As would be recognized by those skilled in the art, the extent to which the outer shell layer covers the inner core depends, at least in part, on the amount of material for the outer shell layer that is combined with a particular amount of material for the inner core. In one embodiment, the core material comprises cationic or cationically ionizable lipid or lipid-like material and the shell material comprises cationic polymer. In one embodiment, the core material comprises cationic polymer and the shell material comprises cationic or cationically ionizable lipid or lipid-like material.

The term “agitate” generally relates to applying mechanical stress. Agitation may comprises one or more selected from the group consisting of stirring, sonification, extrusion, centrifugation such as dual asymmetric centrifugation, vortexing and compounding.

The term “organic solvent” relates to a composition comprising an organic solvent, e.g., chloroform, ethanol, dichloromethane, or a mixture of organic solvents.

The term “average diameter” refers to the mean hydrodynamic diameter of particles as measured by dynamic laser light scattering (DLS) with data analysis using the so-called cumulant algorithm, which provides as results the so-called Z_{average} with the dimension of a length, and the polydispersity index (PI or PDI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

The “polydispersity index” is preferably calculated based on dynamic light scattering measurements by the so-called cumulant analysis as mentioned in the definition of the “average diameter”. Under certain prerequisites, it can be taken as a measure of the size distribution of an ensemble of nanoparticles.

The term “extruding” or “extrusion” refers to the creation of particles having a fixed, cross-sectional profile. In particular, it refers to the downsizing of a particle, whereby the particle is forced through filters with defined pores.

The term “net charge” relates to the total sum of charges, such as positive and negative charges. For example, if a particle comprises a higher number of negative charges than positive charges, the net charge of the particle is negative. If the particle comprises a higher number of positive charges than negative charges, the net charge of the particle is positive. If the particle comprises an equal number of positive charges and negative charges, the net charge of the particle is neutral, particularly electroneutral. Thus, the net charge of a particle according to the present disclosure can be negative, positive or neutral. In one embodiment, the net charge of the particle is positive. In one embodiment, the net charge of the particle is negative.

Terms as “charged”, “net charge”, “negatively charged” or “positively charged” refer to the electric net charge of the given compound or particle when dissolved or suspended in aqueous buffer at the relevant pH (e.g., 7.1).

According to the present disclosure, the terms “N/P ratio”, “NP ratio”, “N:P ratio”, “N/P” and “NP” refer to the molar ratio of nitrogen atoms (N) in the cationic polymer and/or cationic or cationically ionizable lipid or lipid-like material to phosphor atoms (P) in the RNA.

### RNA-Containing Particles

Different types of RNA containing particles have been described previously to be suitable for delivery of RNA in particulate form (e.g. Kaczmarek, J. C. et al., 2017, Genome Medicine 9, 60). For non-viral RNA delivery vehicles, nanoparticle encapsulation of RNA physically protects RNA from degradation and, depending on the specific chemistry, can aid in cellular uptake and endosomal escape.

The present disclosure describes particles comprising RNA, at least one cationic or cationically ionizable lipid or lipid-like material, and at least one cationic polymer which associate with RNA to form RNA particles and compositions comprising such particles. The RNA particles may comprise RNA which is complexed in different forms by non-covalent interactions to the particle. The particles described herein are not viral particles, in particular infectious viral particles, i.e., they are not able to virally infect cells.

Generally, the RNA particles described herein are obtainable by adding RNA to a colloidal dispersion comprising at least one cationic or cationically ionizable lipid or lipid-like material and at least one cationic polymer. Suitable cationic or cationically ionizable lipids or lipid-like materials and cationic polymers are those that form such colloidal dispersions and RNA particles and are included by the term “particle forming components” or “particle forming agents”. The term “particle forming components” or “particle forming agents” relates to any components which associate with RNA to form RNA particles. Such components include any component which can be part of RNA particles.

### Cationic Polymer

Given their high degree of chemical flexibility, polymers are commonly used materials for nanoparticle-based delivery. Typically, cationic polymers are used to electrostatically condense the negatively charged RNA into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine and polyethyleneimine, as well as naturally occurring polymers such as protamine or chitosan have all been applied to RNA delivery and are suitable as cationic polymers herein. In addition, some investigators have synthesized polymers specifically for nucleic acid delivery. Poly(β-amino esters), in particular, have gained widespread use in nucleic acid delivery owing to their ease of synthesis and biodegradability. Such synthetic polymers are also suitable as cationic polymers herein.

A “polymer”, as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties such as those described herein.

If more than one type of repeat unit is present within the polymer, then the polymer is said to be a “copolymer”. It is to be understood that the polymer being employed herein can be a copolymer. The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, the polymer is biocompatible. Biocompatible polymers are polymers that typically do not result in significant cell death at moderate concentrations. In certain embodiments, the biocompatible polymer is biodegradable, i.e., the polymer is able to degrade, chemically and/or biologically, within a physiological environment, such as within the body.

In certain embodiments, polymer may be protamine or polyalkyleneimine, in particular protamine.

The term “protamine” refers to any of various strongly basic proteins of relatively low molecular weight that are rich in arginine and are found associated especially with DNA in place of somatic histones in the sperm cells of various animals (as fish). In particular, the term “protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

According to the disclosure, the term “protamine” as used herein is meant to comprise any protamine amino acid sequence obtained or derived from natural or biological sources including fragments thereof and multimeric forms of said amino acid sequence or fragment thereof as well as (synthesized) polypeptides which are artificial and specifically designed for specific purposes and cannot be isolated from native or biological sources.

In one embodiment, the polyalkyleneimine comprises polyethylenimine and/or polypropylenimine, preferably polyethyleneimine. A preferred polyalkyleneimine is polyethyleneimine (PEI). The average molecular weight of PEI is preferably 0.75·10² to 10⁷ Da, preferably 1000 to 10⁵ Da, more preferably 10000 to 40000 Da, more preferably 15000 to 30000 Da, even more preferably 20000 to 25000 Da.

Preferred according to the disclosure is linear polyalkyleneimine such as linear polyethyleneimine (PEI).

Cationic polymers (including polycationic polymers) contemplated for use herein include any cationic polymers which are able to electrostatically bind RNA. In one embodiment, cationic polymers contemplated for use herein include any cationic polymers which together with cationic or cationically ionizable lipids or lipid-like materials can form colloids, in particular vesicles, with which RNA can be associated, e.g. by forming complexes with the RNA or forming vesicles in which the RNA is enclosed or encapsulated.

### Lipid and Lipid-Like Material

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids. As used herein, the term “lipid” is to be construed to cover both lipids and lipid-like materials unless otherwise indicated herein or clearly contradicted by context.

Specific examples of amphiphilic compounds that may be included in an amphiphilic layer include, but are not limited to, phospholipids, aminolipids and sphingolipids.

In certain embodiments, the amphiphilic compound is a lipid. The term “lipid” refers to a group of organic compounds that are characterized by being insoluble in water, but soluble in many organic solvents. Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule’s configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

### Cationic or Cationically Ionizable Lipids or Lipid-Like Materials

The RNA particles described herein comprise at least one cationic or cationically ionizable lipid or lipid-like material as particle forming agent. Cationic or cationically ionizable lipids or lipid-like materials contemplated for use herein include any cationic or cationically ionizable lipids or lipid-like materials which are able to electrostatically bind RNA. In one embodiment, cationic or cationically ionizable lipids or lipid-like materials contemplated for use herein together with cationic polymers can form colloids, in particular vesicles, with which RNA can be associated, e.g. by forming complexes with the RNA or forming vesicles in which the RNA is enclosed or encapsulated.

As used herein, a “cationic lipid” or “cationic lipid-like material” refers to a lipid or lipid-like material having a net positive charge. Cationic lipids or lipid-like materials bind negatively charged RNA by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as neutral pH. This ionizable behavior is thought to enhance efficacy through helping with endosomal escape and reducing toxicity as compared with particles that remain cationic at physiological pH.

For purposes of the present disclosure, such “cationically ionizable” lipids or lipid-like materials are comprised by the term “cationic lipid or lipid-like material” unless contradicted by the circumstances.

In one embodiment, the cationic or cationically ionizable lipid or lipid-like material comprises a head group which includes at least one nitrogen atom (N) which is positive charged or capable of being protonated.

Examples of cationic lipids include, but are not limited to 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N',N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy- N-[2(spermine carboxamide)ethyl]-N,N-dimethyl-l-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5'-(cholest-5-en-3-beta-oxy)-3'-oxapentoxy)-3-dimethyl-1-(cis,cis-9',12'-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (βAE-DMRIE), N-(4-carboxybenzyl)-N,N-dimethyl-2,3-bis(oleoyloxy)propan-1-aminium (DOBAQ), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8'-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N₁₂-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200). Preferred are DOTAP, DODMA, DOTMA, DODAC, and DOSPA. In specific embodiments, the at least one cationic lipid is DOTAP.

In some embodiments, the cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle.

### Additional Lipids or Lipid-Like Materials

Particles described herein may also comprise lipids or lipid-like materials other than cationic or cationically ionizable lipids or lipid-like materials, i.e., non-cationic lipids or lipid-like materials (including non-cationically ionizable lipids or lipid-like materials). Collectively, anionic and neutral lipids or lipid-like materials are referred to herein as non-cationic lipids or lipid-like materials. Optimizing the formulation of RNA particles by addition of other hydrophobic moieties, such as cholesterol and lipids, in addition to an ionizable/cationic lipid or lipid-like material may enhance particle stability and efficacy of RNA delivery.

An additional lipid or lipid-like material may be incorporated which may or may not affect the overall charge of the RNA particles. In certain embodiments, the additional lipid or lipid-like material is a non-cationic lipid or lipid-like material. The non-cationic lipid may comprise, e.g., one or more anionic lipids and/or neutral lipids. As used herein, a “neutral lipid” refers to any of a number of lipid species that exist either in an uncharged or neutral zwitterionic form at a selected pH. In preferred embodiments, the additional lipid comprises one of the following neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2'-hydroxyethyl ether, cholesteryl-4'- hydroxybutyl ether, tocopherol and derivatives thereof, and mixtures thereof.

Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain preferred embodiments, the additional lipid is DSPC or DSPC and cholesterol.

In certain embodiments, the RNA particles include both a cationic lipid and an additional lipid. In an exemplary embodiment, the cationic lipid is DOTAP and the additional lipid is DSPC or DSPC and cholesterol.

Without wishing to be bound by theory, the amount of the at least one cationic lipid compared to the amount of the at least one additional lipid may affect important RNA particle characteristics, such as charge, particle size, stability, tissue selectivity, and bioactivity of the RNA. Accordingly, in some embodiments, the molar ratio of the at least one cationic lipid to the at least one additional lipid is from about 10:0 to about 1:9, about 4:1 to about 1:2, or about 3:1 to about 1:1.

In some embodiments, the non-cationic lipid, in particular neutral lipid, (e.g., one or more phospholipids and/or cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in the particle.

### Targeting Agents

One or more of the particle-forming components described herein such as polymers, lipids or lipid-like materials may comprise or may be functionalized with one or more molecular moieties conferring certain properties, such as positive or negative charge, or a targeting agent that will direct the particle to a particular cell type, collection of cells, or tissue.

A variety of suitable targeting agents are known in the art. Non-limiting examples of targeting agents include a peptide, a protein, an enzyme, a nucleic acid, a fatty acid, a hormone, an antibody, a carbohydrate, mono-, oligo- or polysaccharides, a peptidoglycan, a glycopeptide, or the like. For example, any of a number of different materials that bind to antigens on the surfaces of target cells can be employed. Antibodies to target cell surface antigens will generally exhibit the necessary specificity for the target. In addition to antibodies, suitable immunoreactive fragments can also be employed, such as the Fab, Fab', F(ab')2 or scFv fragments or single-domain antibodies (e.g. camelids V_{H}H fragments). Many antibody fragments suitable for use in forming the targeting mechanism are already available in the art. Similarly, ligands for any receptors on the surface of the target cells can suitably be employed as targeting agent. These include any small molecule or biomolecule, natural or synthetic, which binds specifically to a cell surface receptor, protein or glycoprotein found at the surface of the desired target cell.

### RNA Particles

A “RNA particle” includes a formulation that can be used to deliver RNA to a target site of interest (e.g., cell, tissue, organ, and the like). A RNA particle herein is formed from at least one cationic or cationically ionizable lipid or lipid-like material such as DOTAP, at least one cationic polymer such as protamine, and RNA.

Without intending to be bound by any theory, it is believed that the cationic or cationically ionizable lipid or lipid-like material and the cationic polymer combine together with the RNA to form aggregates, and this aggregation results in colloidally stable particles.

In some embodiments, RNA particles comprise more than one type of RNA molecules, where the molecular parameters of the RNA molecules may be similar or different from each other, like with respect to molar mass or fundamental structural elements such as molecular architecture, capping, coding regions or other features,

The RNA particles described herein in addition to RNA comprise (i) a cationic or cationically ionizable lipid or lipid-like material, and (ii) a cationic polymer. The cationic or cationically ionizable lipid or lipid-like material may comprise from about 10 mol % to about 100 mol %, from about 20 mol % to about 100 mol %, from about 30 mol % to about 100 mol %, from about 40 mol % to about 100 mol %, or from about 50 mol % to about 100 mol % of the total lipid present in the particle. The molar ratio of the number of nitrogen atoms (N) in the cationic or cationically ionizable lipid or lipid-like material and cationic polymer to the number of phosphor atoms (P) in the RNA (N/P ratio) is preferably 1.0 to 10.0, preferably 1.2 to 5.0, 1.5 to 2.5 or about 2.0.

In certain embodiments of the present disclosure, the cationic or cationically ionizable lipid or lipid-like material in the RNA particles described herein is at a concentration from about 0.02 mg/mL to about 5 mg/mL, from about 0.02 mg/mL to about 2 mg/mL, from about 0.05 mg/mL to about 2 mg/mL, or from about 0.1 mg/mL to about 1 mg/mL.

In certain embodiments of the present disclosure, the cationic polymer in the RNA particles described herein is at a concentration from about 0.002 mg/mL to about 5 mg/mL, from about 0.002 mg/mL to about 2 mg/mL, from about 0.005 mg/mL to about 2 mg/mL, from about 0.01 mg/mL to about 1 mg/mL, or from about 0.05 mg/mL to about 0.5 mg/mL.

### RNA Particle Diameter

RNA particles described herein have an average diameter that in one embodiment ranges from about 30 nm to about 1000 nm, from about 50 nm to about 800 nm, from about 70 nm to about 600 nm, from about 90 nm to about 400 nm, or from about 100 nm to about 300 nm.

RNA particles described herein, e.g. generated by the processes described herein, exhibit a polydispersity index less than about 0.5, less than about 0.4, less than about 0.3, or about 0.2 or less. By way of example, the RNA particles can exhibit a polydispersity index in a range of about 0.1 to about 0.3 or about 0.2 to about 0.3.

### RNA Particle Formation

RNA particles described herein can be prepared using a wide range of methods that may involve obtaining a colloid from a mixture comprising at least one cationic or cationically ionizable lipid or lipid-like material and at least one cationic polymer and mixing the colloid with RNA to obtain RNA particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid or lipid-like material and at least one cationic polymer methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

According to the present disclosure, a colloid may be prepared by a. obtaining a mixture comprising at least one cationic or cationically ionizable lipid or lipid-like material in an organic solvent; b. removing the organic solvent from the mixture obtained in step a. thus obtaining a lipid film; c. adding a solution, e.g., an aqueous solution, comprising at least one cationic polymer to the lipid film obtained in step b.; and d. agitating the mixture obtained in step c. to obtain a colloid. Organic solvents or phases that may be used herein include, but are not limited to, chloroform or chloroform:methanol mixtures.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

Accordingly, according to the present disclosure, an aqueous phase containing cationic polymer and an organic phase containing cationic or cationically ionizable lipid or lipid-like material may be mixed and a colloid obtained from this mixture, optionally following removal of the organic phase.

Other methods having organic solvent free characteristics may also be used according to the present disclosure for preparing a colloid.

### RNA

In the present disclosure, the term “RNA” relates to nucleic acid molecules which include ribonucleotide residues. In preferred embodiments, the RNA contains all or a majority of ribonucleotide residues. As used herein, “ribonucleotide” refers to a nucleotide with a hydroxyl group at the 2'-position of a P-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered RNAs are considered analogs of naturally-occurring RNA. In particular embodiments, the RNA according to the disclosure comprises a population of different RNA molecules, e.g. a mixture of different RNA molecules optionally encoding different peptides and/or proteins. Thus, according to the disclosure, the term “RNA” may include a mixture of RNA molecules.

In certain embodiments of the present disclosure, the RNA is messenger RNA (mRNA) that relates to a RNA transcript which encodes a peptide or protein. As established in the art, mRNA generally contains a 5' untranslated region (5'-UTR), a peptide coding region and a 3' untranslated region (3'-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides.

In one embodiment, RNA is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In certain embodiments of the present disclosure, the RNA is replicon RNA or simply “a replicon”, in particular self-replicating RNA. In one particularly preferred embodiment, the replicon or self-replicating RNA is derived from or comprises elements derived from a ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see Jose et al., Future Microbiol., 2009, vol. 4, pp. 837-856). The total genome length of many alphaviruses typically ranges between 11,000 and 12,000 nucleotides, and the genomic RNA typically has a 5'-cap, and a 3’ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5' terminus of the genome, while alphavirus structural proteins are encoded together by a second ORF which is found downstream of the first ORF and extends near the 3’ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234). Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

In certain embodiments of the present disclosure, the RNA in the RNA particles described herein is at a concentration from about 0.002 mg/mL to about 5 mg/mL, from about 0.002 mg/mL to about 2 mg/mL, from about 0.005 mg/mL to about 2 mg/mL, from about 0.01 mg/mL to about 1 mg/mL, from about 0.05 mg/mL to about 0.5 mg/mL or from about 0.1 mg/mL to about 0.5 mg/mL.

In one embodiment, the RNA may have modified ribonucleotides. Examples of modified ribonucleotides include, without limitation, 5-methylcytidine, pseudouridine (ψ), N1-methyl-pseudouridine (m¹ψ) or 5-methyl-uridine (m⁵U).

In some embodiments, one or more uridine in the RNA described herein is replaced by a modified nucleoside. In some embodiments, the modified nucleoside is a modified uridine.

In some embodiments, RNA comprises a modified nucleoside in place of at least one uridine. In some embodiments, RNA comprises a modified nucleoside in place of each uridine.

In some embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, the modified nucleoside replacing one or more uridine in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(τm5s2U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2'-O-methyl-uridine (Um), 5,2'-O-dimethyl-uridine (m⁵Um), 2'-O-methyl-pseudouridine (ψm), 2-thio-2'-O-methyl-uridine (s²Um), 5-methoxycarbonylmethyl-2'-O-methyl-uridine (mcm⁵Um), 5-carbamoylmethyl-2'-O-methyl-uridine (ncm⁵Um), 5-carboxymethylaminomethyl-2'-O-methyl-uridine (cmnm⁵Um), 3,2'-O-dimethyl-uridine (m³Um), 5-(isopentenylaminomethyl)-2'-O-methyl-uridine (inm⁵Um), 1-thio-uridine, deoxythymidine, 2'-F-ara-uridine, 2'-F-uridine, 2'-OH-ara-uridine, 5-(2-carbomethoxyvinyl) uridine, 5-[3-(1-E-propenylamino)uridine, or any other modified uridine known in the art.

In some embodiments, the RNA according to the present disclosure comprises a 5’-cap. In one embodiment, the RNA of the present disclosure does not have uncapped 5'-triphosphates. In one embodiment, the RNA may be modified by a 5'- cap analog. The term “5'-cap” refers to a structure found on the 5'-end of an mRNA molecule and generally consists of a guanosine nucleotide connected to the mRNA via a 5' to 5' triphosphate linkage. In one embodiment, this guanosine is methylated at the 7-position. Providing an RNA with a 5'-cap or 5'-cap analog may be achieved by in vitro transcription, in which the 5'-cap is co-transcriptionally expressed into the RNA strand, or may be attached to RNA post-transcriptionally using capping enzymes.

In some embodiments, RNA according to the present disclosure comprises a 5’-UTR and/or a 3’-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5’ (upstream) of an open reading frame (5'-UTR) and/or 3’ (downstream) of an open reading frame (3’-UTR). A 5’-UTR, if present, is located at the 5' end, upstream of the start codon of a protein-encoding region. A 5’-UTR is downstream of the 5'-cap (if present), e.g. directly adjacent to the 5’-cap. A 3'-UTR, if present, is located at the 3' end, downstream of the termination codon of a protein-encoding region, but the term “3'-UTR” does preferably not include the poly(A) tail. Thus, the 3’-UTR is upstream of the poly(A) sequence (if present), e.g. directly adjacent to the poly(A) sequence.

In some embodiments, the RNA according to the present disclosure comprises a 3'-poly(A) sequence. As used herein, the term “poly(A) sequence” or “poly-A tail” refers to an uninterrupted or interrupted sequence of adenylate residues which is typically located at the 3' end of an RNA molecule. Poly(A) sequences are known to those of skill in the art and may follow the 3' UTR in the RNAs described herein. The poly(A) sequence may be of any length. In some embodiments, a poly(A) sequence comprises or consists of at least 20, at least 30, at least 40, at least 80, or at least 100 and up to 500, up to 400, up to 300, up to 200, or up to 150 nucleotides, and, in particular, about 110 nucleotides. In some embodiments, the poly(A) sequence only consists of A nucleotides. In some embodiments, the poly(A) sequence essentially consists of A nucleotides, but is interrupted by a random sequence of the four nucleotides (A, C, G, and U), as disclosed in WO 2016/005324 A1, hereby incorporated by reference. Such random sequence may be 5 to 50, 10 to 30, or 10 to 20 nucleotides in length. A poly(A) cassette present in the coding strand of DNA that essentially consists of dA nucleotides, but is interrupted by a random sequence having an equal distribution of the four nucleotides (dA, dC, dG, dT) and having a length of e.g. 5 to 50 nucleotides shows, on DNA level, constant propagation of plasmid DNA in E. coli and is still associated, on RNA level, with the beneficial properties with respect to supporting RNA stability and translational efficiency. In some embodiments, no nucleotides other than A nucleotides flank a poly(A) sequence at its 3' end, i.e., the poly(A) sequence is not masked or followed at its 3' end by a nucleotide other than A. In the context of the present disclosure, the term “transcription” relates to a process, wherein the genetic code in a DNA sequence is transcribed into RNA. Subsequently, the RNA may be translated into peptide or protein.

With respect to RNA, the term “expression” or “translation” relates to the process in the ribosomes of a cell by which a strand of mRNA directs the assembly of a sequence of amino acids to make a peptide or protein.

RNA can be coding RNA, i.e. RNA encoding a peptide or protein. Said RNA may express the encoded peptide or protein. For example, said RNA may be RNA encoding and expressing a pharmaceutically active peptide or protein. Alternatively, the RNA can be non-coding RNA such as antisense-RNA, micro RNA (miRNA) or siRNA.

RNA used herein may be pharmaceutically active RNA. A “pharmaceutically active RNA” is a RNA that encodes a pharmaceutically active peptide or protein or is pharmaceutically active in its own, e.g., it has one or more pharmaceutical activities such as those described for pharmaceutically active proteins, e.g., immunostimulatory activity. For example, the RNA may be one or more strands of RNA interference (RNAi). Such agents include short interfering RNAs (siRNAs), or short hairpin RNAs (shRNAs), or precursor of a siRNA or microRNA-like RNA, targeted to a target transcript, e.g., a transcript of an endogenous disease-related transcript of a subject.

Some aspects of the disclosure involve the targeted delivery of the RNA disclosed herein to certain cells or tissues. In one embodiment, the disclosure involves targeting the lymphatic system, in particular secondary lymphoid organs, more specifically spleen. Targeting the lymphatic system, in particular secondary lymphoid organs, more specifically spleen is in particular preferred if the RNA administered is RNA encoding an antigen or epitope for inducing an immune response. In one embodiment, the target cell is a spleen cell. In one embodiment, the target cell is an antigen presenting cell such as a professional antigen presenting cell in the spleen. In one embodiment, the target cell is a dendritic cell in the spleen. The “lymphatic system” is part of the circulatory system and an important part of the immune system, comprising a network of lymphatic vessels that carry lymph. The lymphatic system consists of lymphatic organs, a conducting network of lymphatic vessels, and the circulating lymph. The primary or central lymphoid organs generate lymphocytes from immature progenitor cells. The thymus and the bone marrow constitute the primary lymphoid organs. Secondary or peripheral lymphoid organs, which include lymph nodes and the spleen, maintain mature naive lymphocytes and initiate an adaptive immune response.

Lipid-based RNA delivery systems have an inherent preference to the liver. Liver accumulation is caused by the discontinuous nature of the hepatic vasculature or the lipid metabolism (liposomes and lipid or cholesterol conjugates). In one embodiment, the target organ is liver and the target tissue is liver tissue. The delivery to such target tissue is preferred, in particular, if presence of RNA or of the encoded peptide or protein in this organ or tissue is desired and/or if it is desired to express large amounts of the encoded peptide or protein and/or if systemic presence of the encoded peptide or protein, in particular in significant amounts, is desired or required.

In one embodiment, after administration of the RNA particles described herein, at least a portion of the RNA is delivered to a target cell or target organ. In one embodiment, at least a portion of the RNA is delivered to the cytosol of the target cell. In one embodiment, the RNA is RNA encoding a peptide or protein and the RNA is translated by the target cell to produce the peptide or protein. In one embodiment, the target cell is a cell in the liver. In one embodiment, the target cell is a muscle cell. In one embodiment, the target cell is an endothelial cell. In one embodiment the target cell is a tumor cell or a cell in the tumor microenvironment. In one embodiment, the target cell is a blood cell. In one embodiment, the target cell is a cell in the lymph nodes. In one embodiment, the target cell is a cell in the lung. In one embodiment, the target cell is a cell in the skin. In one embodiment, the target cell is a spleen cell. In one embodiment, the target cell is an antigen presenting cell such as a professional antigen presenting cell in the spleen. In one embodiment, the target cell is a dendritic cell in the spleen. In one embodiment, the target cell is a T cell. In one embodiment, the target cell is a B cell. In one embodiment, the target cell is a NK cell. In one embodiment, the target cell is a monocyte. Thus, RNA particles described herein may be used for delivering RNA to such target cell. Accordingly, the present disclosure also relates to a method for delivering RNA to a target cell in a subject comprising the administration of the RNA particles described herein to the subject. In one embodiment, the RNA is delivered to the cytosol of the target cell. In one embodiment, the RNA is RNA encoding a peptide or protein and the RNA is translated by the target cell to produce the peptide or protein.

In an embodiment, RNA encodes a pharmaceutically active peptide or protein.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

According to the disclosure, the term “RNA encodes” means that the RNA, if present in the appropriate environment, such as within cells of a target tissue, can direct the assembly of amino acids to produce the peptide or protein it encodes during the process of translation. In one embodiment, RNA is able to interact with the cellular translation machinery allowing translation of the peptide or protein. A cell may produce the encoded peptide or protein intracellularly (e.g. in the cytoplasm), may secrete the encoded peptide or protein, or may produce it on the surface.

According to the disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” refers to large peptides, in particular peptides having at least about 151 amino acids, but the terms “peptide” and “protein” are used herein usually as synonyms.

A “pharmaceutically active peptide or protein” or “therapeutic peptide or protein” has a positive or advantageous effect on a condition or disease state of a subject when provided to the subject in a therapeutically effective amount. In one embodiment, a pharmaceutically active peptide or protein has curative or palliative properties and may be administered to ameliorate, relieve, alleviate, reverse, delay onset of or lessen the severity of one or more symptoms of a disease or disorder. A pharmaceutically active peptide or protein may have prophylactic properties and may be used to delay the onset of a disease or to lessen the severity of such disease or pathological condition. The term “pharmaceutically active peptide or protein” includes entire proteins or polypeptides, and can also refer to pharmaceutically active fragments thereof. It can also include pharmaceutically active analogs of a peptide or protein.

Examples of pharmaceutically active proteins include, but are not limited to, cytokines and derivatives thereof such as cytokine-fusions (like albumin-cytokine fusions) and immune system proteins such as immunologically active compounds (e.g., interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, seletins, homing receptors, T cell receptors, chimeric antigen receptors (CARs), immunoglobulins including antibodies or bispecific antibodies, e.g., for immune stimulation or production of neutralizing antibodies in case of viral/bacterial infection, soluble major histocompatibility complex antigens, immunologically active antigens such as bacterial, parasitic, or viral antigens, allergens, autoantigens, antibodies), hormones (insulin, thyroid hormone, catecholamines, gonadotrophines, trophic hormones, prolactin, oxytocin, dopamine, bovine somatotropin, leptins and the like), growth hormones (e.g., human grown hormone), growth factors (e.g., epidermal growth factor, nerve growth factor, insulin-like growth factor and the like), growth factor receptors, enzymes (tissue plasminogen activator, streptokinase, cholesterol biosynthestic or degradative, steriodogenic enzymes, kinases, phosphodiesterases, methylases, de-methylases, dehydrogenases, cellulases, proteases, lipases, phospholipases, aromatases, cytochromes, adenylate or guanylaste cyclases, neuramidases, lysosomal enzymes and the like), receptors (steroid hormone receptors, peptide receptors), binding proteins (growth hormone or growth factor binding proteins and the like), transcription and translation factors, tumor growth suppressing proteins (e.g., proteins which inhibit angiogenesis), structural proteins (such as collagen, fibroin, fibrinogen, elastin, tubulin, actin, and myosin), blood proteins (thrombin, serum albumin, Factor VII, Factor VIII, insulin, Factor IX, Factor X, tissue plasminogen activator, protein C, Von Willebrand factor, antithrombin III, glucocerebrosidase, erythropoietin granulocyte colony stimulating factor (GCSF) or modified Factor VIII, anticoagulants and the like.

The term “immunologically active compound” relates to any compound altering an immune response, for example, by inducing and/or suppressing maturation of immune cells, inducing and/or suppressing cytokine biosynthesis, and/or altering humoral immunity by stimulating antibody production by B cells. Immunologically active compounds possess potent immunostimulating activity including, but not limited to, antiviral and antitumoral activity, and can also down-regulate other aspects of the immune response, for example shifting the immune response away from a TH2 immune response, which is useful for treating a wide range of TH2 mediated diseases. Immunologically active compounds can be useful as vaccine adjuvants.

In one embodiment, a pharmaceutically active peptide or protein comprises a cytokine. The term “cytokine” refers to a category of small proteins (~5-20 kDa) that are important in cell signalling. Their release has an effect on the behavior of cells around them. Cytokines are involved in autocrine signalling, paracrine signalling and endocrine signalling as immunomodulating agents. Cytokines include chemokines, interferons, interleukins, lymphokines, and tumour necrosis factors but generally not hormones or growth factors (despite some overlap in the terminology). Cytokines are produced by a broad range of cells, including immune cells like macrophages, B lymphocytes, T lymphocytes and mast cells, as well as endothelial cells, fibroblasts, and various stromal cells. A given cytokine may be produced by more than one type of cell. Cytokines act through receptors, and are especially important in the immune system; cytokines modulate the balance between humoral and cell-based immune responses, and they regulate the maturation, growth, and responsiveness of particular cell populations. Some cytokines enhance or inhibit the action of other cytokines in complex ways. In one embodiment, the pharmaceutically active protein according to the disclosure is a cytokine which is involved in regulating lymphoid homeostasis, preferably a cytokine which is involved in and preferably induces or enhances development, priming, expansion, differentiation and/or survival of T cells. In one embodiment, the cytokine is an interleukin. In one embodiment, the pharmaceutically active protein according to the disclosure is an interleukin selected from the group consisting of IL-2, IL-7, IL-12, IL-15, and IL-21.

In one embodiment, a pharmaceutically active peptide or protein comprises a replacement protein. In this embodiment, the present disclosure provides a method for treatment of a subject having a disorder requiring protein replacement (e.g., protein deficiency disorders) comprising administering to the subject RNA as described herein encoding a replacement protein. The term “protein replacement” refers to the introduction of a protein (including functional variants thereof) into a subject having a deficiency in such protein. The term also refers to the introduction of a protein into a subject otherwise requiring or benefiting from providing a protein, e.g., suffering from protein insufficiency. The term “disorder characterized by a protein deficiency” refers to any disorder that presents with a pathology caused by absent or insufficient amounts of a protein. This term encompasses protein folding disorders, i.e., conformational disorders, that result in a biologically inactive protein product. Protein insufficiency can be involved in infectious diseases, immunosuppression, organ failure, glandular problems, radiation illness, nutritional deficiency, poisoning, or other environmental or external insults.

In one embodiment, a pharmaceutically active peptide or protein comprises one or more antigens or one or more epitopes, i.e., administration of the peptide or protein to a subject elicits an immune response against the one or more antigens or one or more epitopes in a subject which may be therapeutic or partially or fully protective.

The term “antigen” relates to an agent comprising an epitope against which an immune response can be generated. The term “antigen” includes, in particular, proteins and peptides. In one embodiment, an antigen is presented by cells of the immune system such as antigen presenting cells like dendritic cells or macrophages. An antigen or a processing product thereof such as a T cell epitope is in one embodiment bound by a T or B cell receptor, or by an immunoglobulin molecule such as an antibody. Accordingly, an antigen or a processing product thereof may react specifically with antibodies or T-lymphocytes (T-cells). In one embodiment, an antigen is a disease-associated antigen, such as a tumor antigen, a viral antigen, or a bacterial antigen and an epitope is derived from such antigen.

The term “disease-associated antigen” is used in its broadest sense to refer to any antigen associated with a disease. A disease-associated antigen is a molecule which contains epitopes that will stimulate a host’s immune system to make a cellular antigen-specific immune response and/or a humoral antibody response against the disease. The disease-associated antigen or an epitope thereof may therefore be used for therapeutic purposes. Disease-associated antigens may be associated with infection by microbes, typically microbial antigens, or associated with cancer, typically tumors.

The term “tumor antigen” refers to a constituent of cancer cells which may be derived from the cytoplasm, the cell surface and the cell nucleus. In particular, it refers to those antigens which are produced intracellularly or as surface antigens on tumor cells.

The term “viral antigen” refers to any viral component having antigenic properties, i.e. being able to provoke an immune response in an individual. The viral antigen may be a viral ribonucleoprotein or an envelope protein.

The term “bacterial antigen” refers to any bacterial component having antigenic properties, i.e. being able to provoke an immune response in an individual. The bacterial antigen may be derived from the cell wall or cytoplasm membrane of the bacterium.

The term “epitope” refers to a part or fragment a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 10 and about 25 amino acids in length, for example, the epitope may be preferably 9, 10, 11, 12, 13, 14, 15, 16, 17, 18, 19, 20, 21, 22, 23, 24, or 25 amino acids in length. In one embodiment, an epitope is between about 10 and about 25 amino acids in length. The term “epitope” includes T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

The terms “T cell” and “T lymphocyte” are used interchangeably herein and include T helper cells (CD4+ T cells) and cytotoxic T cells (CTLs, CD8+ T cells) which comprise cytolytic T cells. The term “antigen-specific T cell” or similar terms relate to a T cell which recognizes the antigen to which the T cell is targeted, in particular when presented on the surface of antigen presenting cells or diseased cells such as cancer cells in the context of MHC molecules and preferably exerts effector functions of T cells. T cells are considered to be specific for antigen if the cells kill target cells expressing an antigen. T cell specificity may be evaluated using any of a variety of standard techniques, for example, within a chromium release assay or proliferation assay. Alternatively, synthesis of lymphokines (such as interferon-y) can be measured. In certain embodiments of the present disclosure, the RNA encodes at least one epitope.

In certain embodiments, the epitope is derived from a tumor antigen. The tumor antigen may be a “standard” antigen, which is generally known to be expressed in various cancers. The tumor antigen may also be a “neo-antigen”, which is specific to an individual’s tumor and has not been previously recognized by the immune system. A neo-antigen or neo-epitope may result from one or more cancer-specific mutations in the genome of cancer cells resulting in amino acid changes. Examples of tumor antigens include, without limitation, p53, ART-4, BAGE, beta-catenin/m, Bcr-abL CAMEL, CAP-1 , CASP-8, CDC27/m, CDK4/m, CEA, the cell surface proteins of the claudin family, such as CLAUD ΓN-6, CLAUDIN-18.2 and CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1 , MAGE-A2, MAGE- A3, MAGE-A4, MAGE- A5, MAGE-A6, MAGE-A7, MAGE-A8, MAGE-A9, MAGE-A 10, MAGE-A 1 1, or MAGE- A12, MAGE-B, MAGE-C, MART- 1 /Melan-A, MC1R, Myosin/m, MUC1 , MUM-1 , MUM -2, MUM -3, NA88-A, NF1 , NY-ESO-1 , NY-BR-1 , pl90 minor BCR-abL, Pml/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1 , TPI/m, TRP-1 , TRP-2, TRP-2/INT2, TPTE, WT, and WT-1.

Cancer mutations vary with each individual. Thus, cancer mutations that encode novel epitopes (neo-epitopes) represent attractive targets in the development of vaccine compositions and immunotherapies. The efficacy of tumor immunotherapy relies on the selection of cancer-specific antigens and epitopes capable of inducing a potent immune response within a host. RNA can be used to deliver patient-specific tumor epitopes to a patient. Dendritic cells (DCs) residing in the spleen represent antigen-presenting cells of particular interest for RNA expression of immunogenic epitopes or antigens such as tumor epitopes. The use of multiple epitopes has been shown to promote therapeutic efficacy in tumor vaccine compositions. Rapid sequencing of the tumor mutanome may provide multiple epitopes for individualized vaccines which can be encoded by RNA described herein, e.g., as a single polypeptide wherein the epitopes are optionally separated by linkers. In certain embodiments of the present disclosure, the RNA encodes at least one epitope, at least two epitopes, at least three epitopes, at least four epitopes, at least five epitopes, at least six epitopes, at least seven epitopes, at least eight epitopes, at least nine epitopes, or at least ten epitopes. Exemplary embodiments include RNA that encodes at least five epitopes (termed a “pentatope”), RNA that encodes at least ten epitopes (termed a “decatope”), RNA that encodes at least twenty epitopes (termed a “eicosatope”).

### Charge Ratio

The electric charge of the RNA particles of the present disclosure is the sum of the electric charges present in the at least one cationic or cationically ionizable lipid or lipid-like material, and the at least one cationic polymer and the electric charges present in the RNA. The charge ratio is the ratio of the positive charges present in the at least one cationic or cationically ionizable lipid or lipid-like material, and the at least one cationic polymer to the negative charges present in the RNA.

In a first embodiment, at physiological pH the charge ratio of positive charges to negative charges in the RNA particles is from about 1.0 to about 10.0, preferably from about 1.2 to about 5.0, from about 1.5 to about 2.5 or about 2.0. In one embodiment, the RNA particles described herein may have a net positive charge ratio.

### Compositions Comprising RNA Particles

The term “plurality of RNA particles” refers to a population of a certain number of particles. In certain embodiments, the term refers to a population of more than 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², or 10²³ or more particles.

It will be apparent to those of skill in the art that the plurality of particles can include any fraction of the foregoing ranges or any range therein.

In embodiments, the composition of the present disclosure is a liquid or a solid. Non-limiting examples of a solid include a frozen form, a lyophilized form or a spray-dried form. In a preferred embodiment, the composition is a liquid.

According to the present disclosure, the compositions described herein may comprise salts such as organic or inorganic salts, including, but not limited to, sodium chloride, potassium chloride, dipotassium phosphate, monopotassium phosphate, potassium acetate, potassium bicarbonate, potassium sulfate, potassium acetate, disodium phosphate, monosodium phosphate, sodium acetate, sodium bicarbonate, sodium sulfate, sodium acetate, lithium chloride, magnesium chloride, magnesium phosphate, calcium chloride, and sodium salts of ethylenediaminetetraacetic acid (EDTA) and amino acids.

Compositions described herein may also comprise a stabilizer to avoid substantial loss of the product quality and, in particular, substantial loss of RNA activity during storage, freezing, lyophilization and/or spray-drying, for example to reduce or prevent aggregation, particle collapse, RNA degradation and/or other types of damage.

In an embodiment, the stabilizer is a cryoprotectant or lyoprotectant.

In an embodiment the stabilizer is a carbohydrate. The term “carbohydrate”, as used herein refers to and encompasses monosaccharides, disaccharides, trisaccharides, oligosaccharides and polysaccharides.

In an embodiment, the stabilizer is an amino acid or a surfactant (e.g. poloxamer).

According to the present disclosure, the RNA particle compositions described herein have a pH suitable for the stability of the RNA particles and, in particular, for the stability of the RNA. In one embodiment, the RNA particle compositions described herein have a pH from about 4.0 to about 8.0, or about 5.0 to about 7.5. Without wishing to be bound by theory, the use of buffer maintains the pH of the composition during manufacturing, storage and use of the composition. In certain embodiments of the present disclosure, the buffer may be sodium bicarbonate, monosodium phosphate, disodium phosphate, monopotassium phosphate, dipotassium phosphate, [tris(hydroxymethyl)methylamino]propanesulfonic acid (TAPS), 2-(Bis(2-hydroxyethyl)amino)acetic acid (Bicine), 2-Amino-2-(hydroxymethyl)propane-1,3-diol (Tris), N-(2-Hydroxy-1,1-bis(hydroxyymethyl)ethyl)glycine (Tricine), 3-[[1,3-dihydroxy-2-(hydroxymethyl)propan-2-yl]amino]-2-hydroxypropane-1-sulfonic acid (TAPSO), 2-[4-(2-hydroxyethyl)piperazin-1-yl]ethanesulfonic acid (HEPES), 2-[[1,3-dihydroxy-2-(hydroxymethyl)propan-2-yl] amino] ethanesulfonic acid (TES), 1,4-piperazinediethanesulfonic acid (PIPES), dimethylarsinic acid, 2-morpholin-4-ylethanesulfonic acid (MES), 3-morpholino-2-hydroxypropanesulfonic acid (MOPSO), or phosphate buffered saline (PBS). Other suitable buffering systems may be acetic acid alone or in a salt, citric acid alone or in a salt, boric acid alone or in a salt and phosphoric acid alone or in a salt, or amino acids and amino acid derivatives.

Certain embodiments of the present disclosure contemplate the use of a chelating agent in a composition described herein. Chelating agents refer to chemical compounds that are capable of forming at least two coordinate covalent bonds with a metal ion, thereby generating a stable, water-soluble complex. Without wishing to be bound by theory, chelating agents reduce the concentration of free divalent ions, which may otherwise induce accelerated RNA degradation in the present disclosure. Examples of suitable chelating agents include, without limitation, ethylenediaminetetraacetic acid (EDTA), a salt of EDTA, desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid (DCTA), diethylenetriaminepentaacetic acid (DTPA), bis(aminoethyl)glycolether-N,N,N′,N′-tetraacetic acid, iminodiacetic acid, citric acid, tartaric acid, fumaric acid, or a salt thereof. In certain embodiments, the chelating agent is EDTA or a salt of EDTA. In an exemplary embodiment, the chelating agent is EDTA disodium dihydrate.

In some embodiments, the EDTA is at a concentration from about 0.05 mM to about 5 mM, from about 0.1 mM to about 2.5 mM or from about 0.25 mM to about 1 mM.

### Pharmaceutical Compositions

The compositions comprising RNA particles described herein are useful as or for preparing pharmaceutical compositions or medicaments for therapeutic or prophylactic treatments.

In one aspect, RNA particles described herein are present in a pharmaceutical composition. In another aspect, a composition described herein is a pharmaceutical composition.

The particles of the present disclosure may be administered in the form of any suitable pharmaceutical composition.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation. In the context of the present disclosure, the pharmaceutical composition comprises RNA particles as described herein.

The pharmaceutical compositions of the present disclosure may comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund’s adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cyctokines, such as monokines, lymphokines, interleukins, chemokines. The chemokines may be IL-1, IL-2, IL-3, IL-4, IL-5, IL-6, IL-7, IL-8, IL-9, IL-10, IL-12, INFa, INF-y, GM-CSF, LT-a. Further known adjuvants are aluminium hydroxide, Freund’s adjuvant or oil such as Montanide® ISA51. Other suitable adjuvants for use in the present disclosure include lipopeptides, such as Pam3Cys, as well as lipophilic components, such as saponins, trehalose-6,6-dibehenate (TDB), monophosphoryl lipid-A (MPL), monomycoloyl glycerol (MMG), or glucopyranosyl lipid adjuvant (GLA).

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the particles or compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the particles or compositions described herein may depend on various of such parameters. In the case that a reaction in a patient is insufficient with an initial dose, higher doses (or effectively higher doses achieved by a different, more localized route of administration) may be used.

The pharmaceutical compositions of the present disclosure may contain salts, buffers, preservatives, and optionally other therapeutic agents. In one embodiment, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In one embodiment, the pharmaceutical composition of the present disclosure includes isotonic saline.

Pharmaceutically acceptable carriers, excipients or diluents for therapeutic use are well known in the pharmaceutical art, and are described, for example, in Remington’s Pharmaceutical Sciences, Mack Publishing Co. (A. R Gennaro edit. 1985).

Pharmaceutical carriers, excipients or diluents can be selected with regard to the intended route of administration and standard pharmaceutical practice.

### Routes of Administration of Pharmaceutical Compositions

In one embodiment, pharmaceutical compositions described herein may be administered intramuscularly, intravenously, intraarterially, subcutaneously, intradermally, dermally, or intratumorally. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the pharmaceutical compositions is formulated for systemic administration. In another preferred embodiment, the systemic administration is by intravenous administration.

### Use of Pharmaceutical Compositions

RNA particles described herein may be used in the therapeutic or prophylactic treatment of various diseases, in particular diseases in which provision of a peptide or protein to a subject results in a therapeutic or prophylactic effect. For example, provision of an antigen or epitope which is derived from a virus may be useful in the treatment of a viral disease caused by said virus. Provision of a tumor antigen or epitope may be useful in the treatment of a cancer disease wherein cancer cells express said tumor antigen. Provision of a functional protein or enzyme may be useful in the treatment of genetic disorder characterized by a dysfunctional protein, for example in lysosomal storage diseases (e.g. Mucopolysaccharidoses) or factor deficiencies. Provision of a cytokine or a cytokine-fusion may be useful to modulate tumor microenvironment.

The term “disease” (also referred to as “disorder” herein) refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one’s perspective on life, and one’s personality.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g. mouse, rat, rabbit, dog, cat, cattle, swine, sheep, horse or primate), or any other non-mammal-animal, including birds (chicken), fish or any other animal species that can be afflicted with or is susceptible to a disease or disorder (e.g., cancer, infectious diseases) but may or may not have the disease or disorder, or may have a need for prophylactic intervention such as vaccination, or may have a need for interventions such as by protein replacement. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject, or an individual or subject at risk of a disease.

In one embodiment of the disclosure, the aim is to provide protection against an infectious disease by vaccination.

In one embodiment of the disclosure, the aim is to provide secreted therapeutic proteins, such as antibodies, bispecific antibodies, cytokines, cytokine fusion proteins, enzymes, to a subject, in particular a subject in need thereof.

In one embodiment of the disclosure, the aim is to provide a protein replacement therapy, such as production of erythropoietin, Factor VII, Von Willebrand factor, β-galactosidase, Alpha-N-acetylglucosaminidase, to a subject, in particular a subject in need thereof.

In one embodiment of the disclosure, the aim is to modulate/reprogram immune cells in the blood.

A person skilled in the art will know that one of the principles of immunotherapy and vaccination is based on the fact that an immunoprotective reaction to a disease is produced by immunizing a subject with an antigen or an epitope, which is immunologically relevant with respect to the disease to be treated. Accordingly, pharmaceutical compositions described herein are applicable for inducing or enhancing an immune response. Pharmaceutical compositions described herein are thus useful in a prophylactic and/or therapeutic treatment of a disease involving an antigen or epitope.

The terms “immunization” or “vaccination” describe the process of administering an antigen to an individual with the purpose of inducing an immune response, for example, for therapeutic or prophylactic reasons.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: Preparation of Particles

### 1. Protamine/mRNA Polyplexes

The mRNA stock solution was diluted to a concentration of 0.5 mg/ml mRNA with H₂O and HBGx5 (5-times concentrated HBGx1) to obtain HBGx1 (20 mM HEPES with 5 % (w/v) D-(+)-Glucose) buffered mRNA solution. A stock solution of protamine was diluted with H₂O and HBGx5 to obtain a specific concentration of protamine in HBGx1 with the same volume of mRNA solution. Protamine solution was then given to the mRNA solution in two steps. After the first step, the mixture was vortexed for 10 seconds and after the second step the systems were centrifuged in the dual asymmetric centrifuge (DAC) for 5 minutes at 3000 rpm.

### 2. Preparation of Lipid Films

DOTAP films were produced from a stock solution (25 mg/ml in chloroform) in twist top vials (0.65 ml). Chloroform was evaporated using a Speed vac SVC 200 for 1 hour at 1 mbar. The lipid films were frozen over night at approximately -14° C. Other lipid films mentioned below were prepared with the same method.

### 3. DOTAP/mRNA Lipoplexes

mRNA solution was mixed as described above. DOTAP films were allowed to thaw for 10 minutes and 75 mg silica beads were added to the twist top vials. The thawed films were hydrated for 10-15 minutes with mRNA solution and centrifuged in the DAC twice for 5 minutes at 3000 rpm. The same volume of HBGx1 was given to the DOTAP/mRNA particles and the resulting solution was vortexed for 10 seconds.

### 4. Hybrid mRNA Lipopolyplexes Comprising DOTAP and Protamine

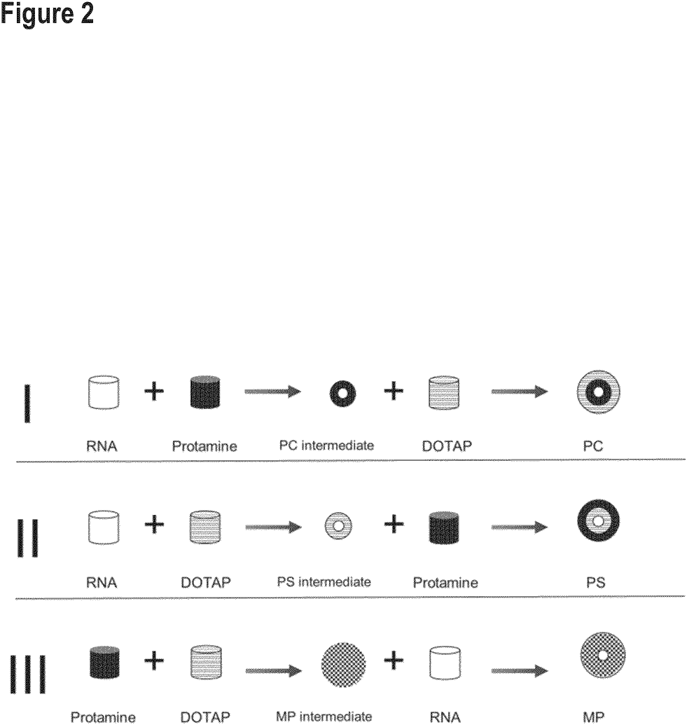

Three different structure morphologies were obtained by variation of the assembly routes (FIG. 2).

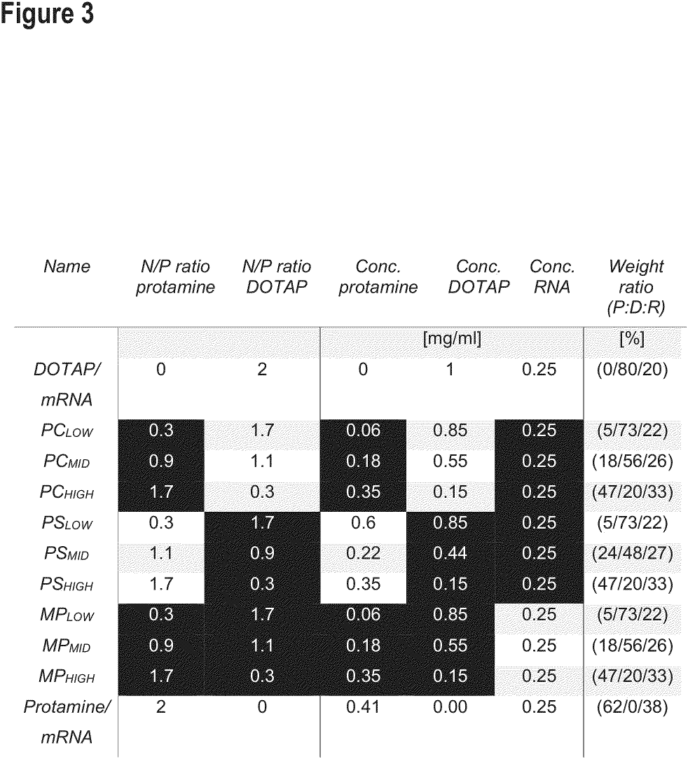

Stock solutions were prepared for luciferase coding mRNA (2.45 mg/ml) and protamine (8 mg/ml) in HEPES buffered glucose (20 mM HEPES with 5 %(w/m) D-(+)-Glucose) at pH 7.0-7.2 and 300 ± 20 mOsmol. The concentration of protamine and DOTAP varied according to the desired proportions of positive charges (15 %, 45 % and 85 % positive charge from protamine). Three different assembly routes were chosen (FIGS. 2, 3), having a negatively charged polymer/mRNA core with a cationic lipid shell (polymer core, PC) or vice versa, having a negatively charged lipid/mRNA core with a cationic polymer shell (polymer shell, PS). The third assembly method was to mix lipid and polymer in advance and enable a self-assembly of the components to form particles with mRNA in a single-step protocol (mixed particles, MP). Particles were prepared using a Phoenix RS-VA10 Vortexer (10 s, 25 W) and a SpeedMixer DAC 150.1 CM 41 (5 min, 3000 rpm). It should be noted that the ratio of PS_{MID} slightly differs to the ratio of PC_{MID} and MP_{MID} - this was necessary in order to obtain negatively charged intermediate particles and to avoid aggregation during manufacturing.

### 4.1 Preparation of PC Particles

200 µl of protamine (variation of concentration) solution in HBGx1 was added to the same volume of mRNA solution in HBGx1. The samples were vortexed for 10 seconds at 25 W and then stored at room temperature for 15 minutes to obtain intermediate particles. In the meantime, DOTAP films were thawed for 10 minutes and 75 mg silica beads were added to the twist top vials. To create final PC particles the intermediate particles were added to the twist top vials in two steps (75 µl and 125 µl) and centrifuged in the dual asymmetric centrifuge (DAC) each time for 5 minutes at 3000 rpm after hydration of the lipid film by the intermediate particle solution for 10-15 minutes.

### 4.2 Preparation of PS Particles

DOTAP films were thawed for 10 minutes and 75 mg silica beads were added to the twist top vials. To form PS intermediate particles, 200 µl mRNA solution was added to the twist top vial in two steps and centrifuged in the DAC for 5 minutes at 3000 rpm after hydration of the lipid film by RNA solution for 10-15 minutes. In order to form the final PS particles, the same volume of protamine solution was added to the intermediate particles and vortexed for 10 seconds at 25 W.

### 4.3 Preparation of MP Particles

The DOTAP films were thawed, silica beads were added, and then 200 µl of protamine solution was added to the twist top vial in two steps (75 µl and 125 µl) and centrifuged in the DAC for 5 minutes at 3000 rpm after hydration of the lipid film by protamine solution for 10-15 minutes. Afterwards, the resulting MP intermediate particles were stored for 15 minutes at room temperature. In a final step, the self-assembled MP intermediate was added to an equal volume of mRNA solution and vortexed for 10 seconds at 35 W to form the final MP particles.

### Example 2: Testing of Particles

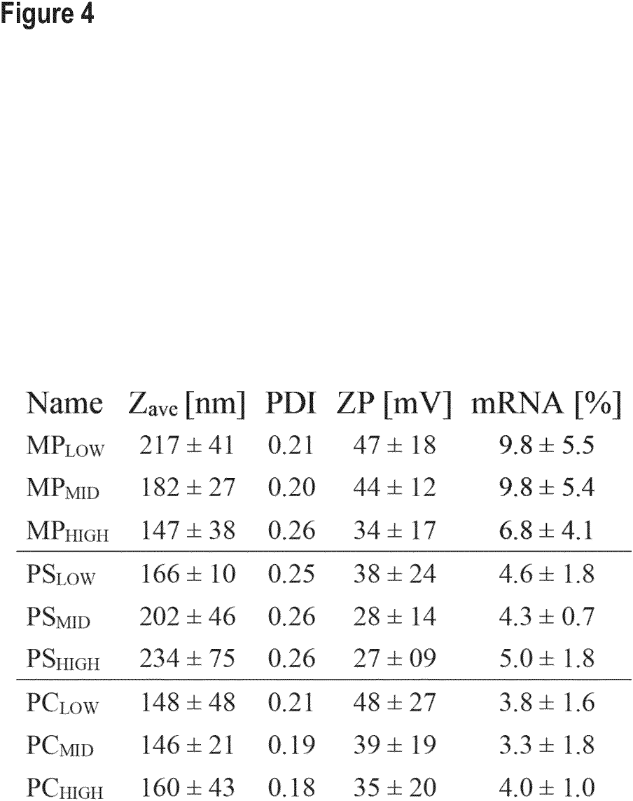

The final particles from the three different assembly sequences (MP, PS, PC) described above, each with three different protamine contributions to the N/P ratio composition (LOW, MID, HIGH) were physicochemically characterized. Values for mean particle size (Zₐᵥₑ), Polydispersity (PDI), zeta potential (ZP) and the percentage of accessible mRNA (mRNA) were obtained from DLS/ELS and Quant-it™ RiboGreen® measurements; see FIG. 4. Each data point resembles the mean value of at least three independent samples measured in triplicates.

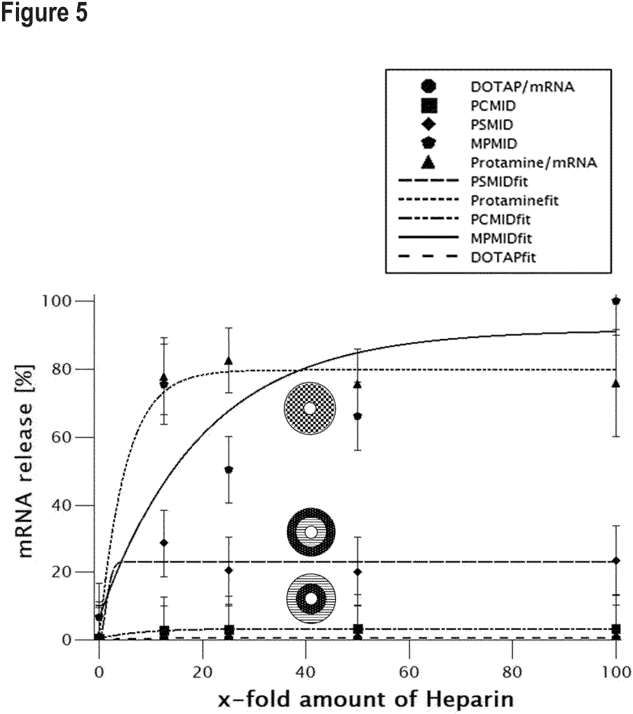

Release of mRNA after addition of heparin in H₂O was tested at an N/P ratio of 2 for protamine/mRNA particles, DOTAP/mRNA particles, MP_{MID}, PC_{MID} and PS_{MID} particles. The curves represent a model fit of the original data (dots). As demonstrated in FIG. 5, the RNA in the mixed particles is released more easily than the one in the particles with core-shell organization.

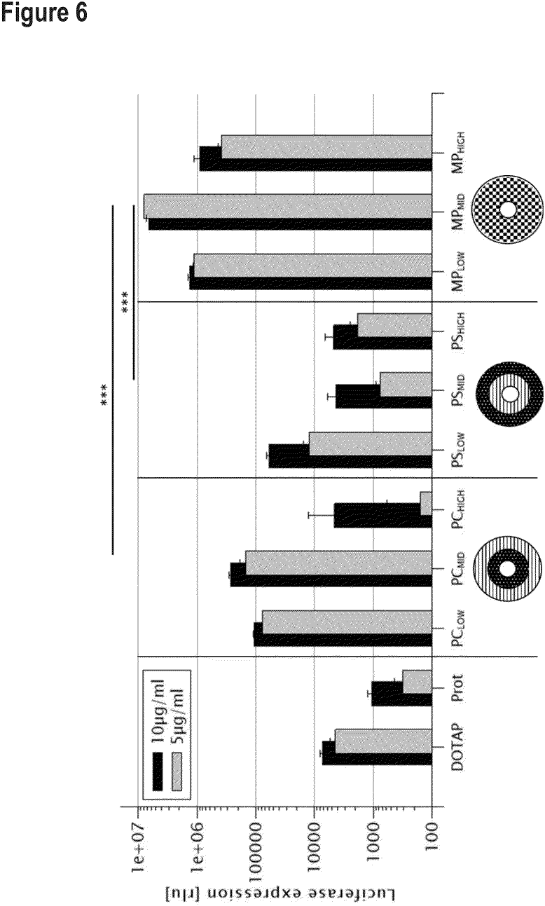

HEK293 cells were transfected with different protamine/DOTAP hybrid nanoparticles at different compositions in comparison to protamine/mRNA and DOTAP/mRNA particles. ***p<0.001. Statistical significance was determined using two-way ANOVA combined with a Bonferroni comparison test. As demonstrated in FIG. 6, the activity of the particles with composite organization is at least one order of magnitude higher than that of the particles with core-shell organization.

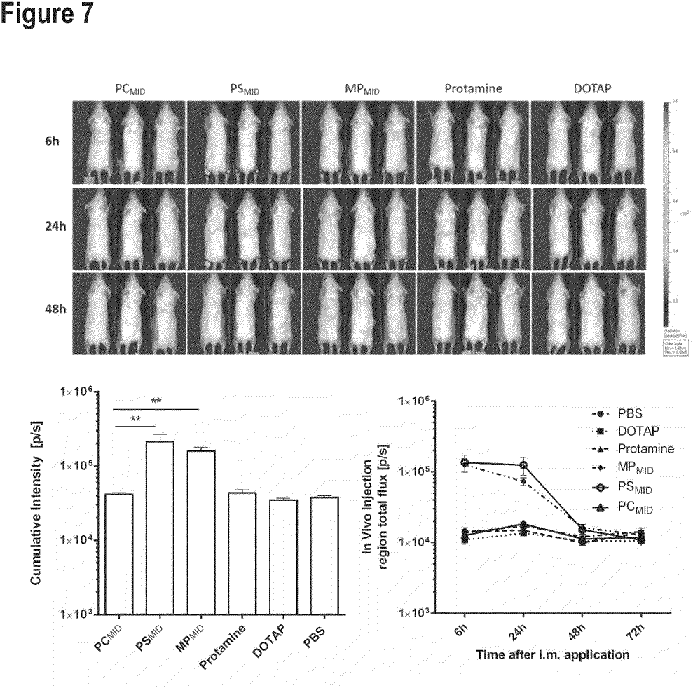

FIG. 7 shows an in vivo evaluation of the particles with the different architectures. Comparing all groups, the particles with composite organization are among those with highest signals. The in vivo experiments show no transfection for PC particles (DOTAP as shell), but only for the single-step assembled hybrid particles (MP) and the particles with protamine as shell (PS). This is in contrast to the in vitro results, where PC particles work better than PS particles. This discrepancy may be due to the better cellular uptake, when DOTAP is at the particle surface, in comparison to a protamine shell (FIG. 6, column 1+2). In animal experiments, however, the PS particles, like the MP particles are more effective. Both systems, show a better mRNA release (heparin release assay, FIG. 5). Due to the diversity of cells present in vivo, the HEK293 cells’ preference for DOTAP in terms of uptake seems not to translate in vivo. Rather, it seems that the release of the mRNA, which is dependent on the particle structure, has the deciding impact on the transfection efficacy during in vivo evaluation. A structural feature of the PS and MP particles seems to enhance transfection in vivo.

### Example 3: Structure of Particles

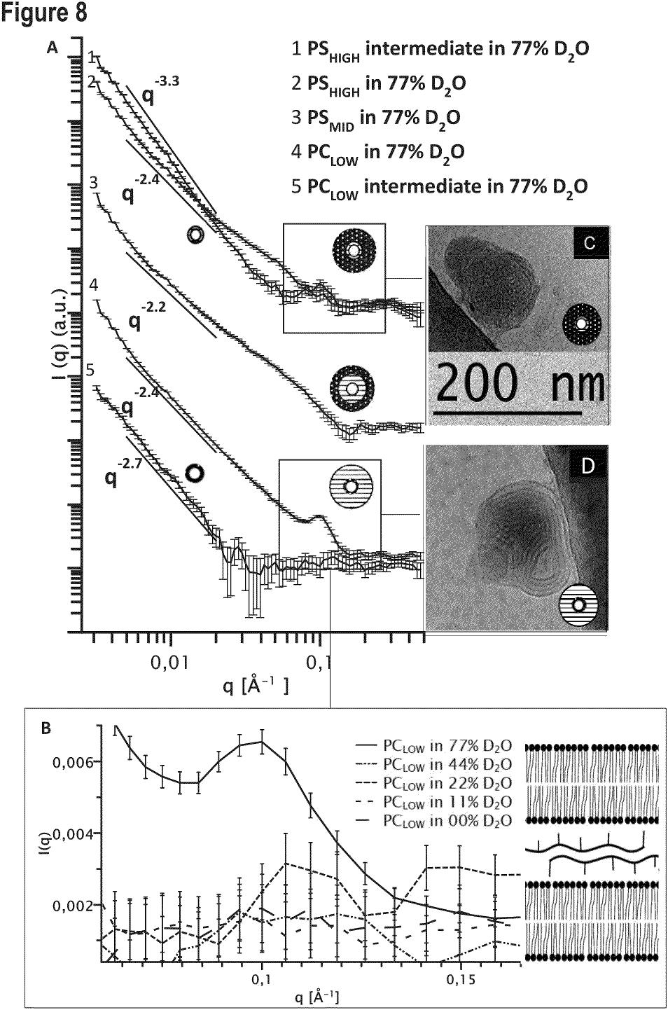

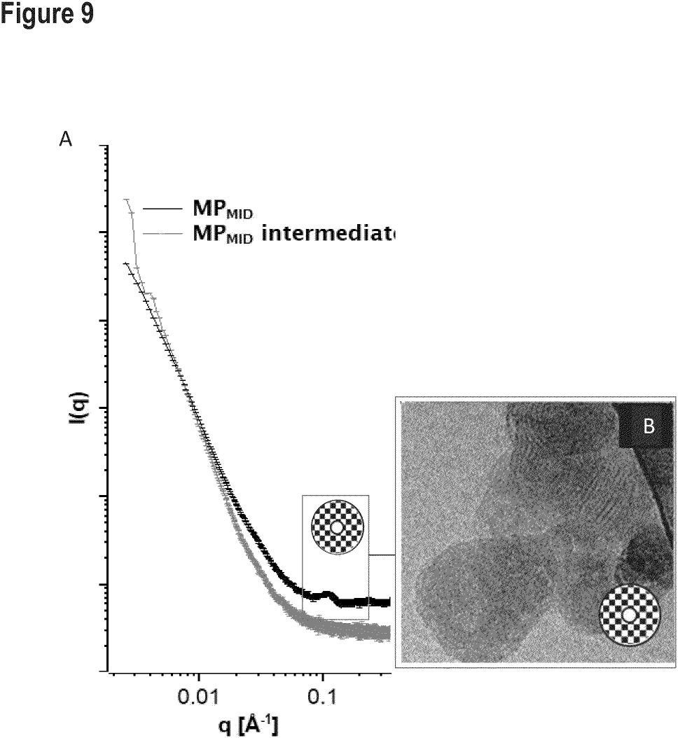

FIG. 8 (A) depict the small angle neutron scattering curve of the intermediate as well as final particles of the sequential assembled PC_{LOW} and PS_{HIGH} particles in 77% D₂O/ 33% H₂O buffer. After background subtraction the scattering curves follow a linear slope of l(q) oc q^{-P} with an exponent of 2<P<3.5 indicating that the particles consist of fractal structures that may be highly polydisperse in size and larger than the resolution limit. The polydispersity can derive from the presence of two different particle populations present in the intermediate state of PC_{LOW} and PS_{HIGH} as mentioned before. Nevertheless, the scattering curves depict structural estimations of high value. Despite a stronger overall intensity (I(q)), the scattering pattern of the final PC_{LOW} particles (polymer/mRNA core with lipid shell) show a peak at a q-value of ~0.1 Å⁻¹ (with a material domain distance of approx. 6.3 nm) that is not present for the intermediate state of PC_{LOW} (polymer/mRNA only). This peak position is in accordance with Bragg peaks observed for comparable systems of cationic liposome oligonucleotide complexes in previous structure-mechanism studies. According to literature, this peak results from an interplay between the negatively charged mRNA backbone and the cationic head groups of DOTAP in which the mRNA is intercalated between the cationic bilayers (FIG. 8(D)). The possibility of adjusting the scattering density of the buffer to match single components makes it possible to make these contributions to the particle ‘transparent’ to neutrons. Therefore, the individual scattering contributions from each component can be deconvolved. In FIG. 8(B) the scattering curves of PC_{LOW} at different D₂O ratios are depicted in detail at q-values between 0.06 and 0.16 Å⁻¹. The peak is only observable at high D₂O concentrations, indicating that the scattering of this reflection derives mainly from a component with a low neutron scattering-length density (NSLD) such as DOTAP. When forming a DOTAP/mRNA intermediate state (PS_{HIGH} intermediate, FIG. 8(A)) the same Bragg peak is present but disappears when adding protamine to form the final PS_{HIGH} particle. Therefore, the formation of a new nanoparticulate system due to addition of protamine can be proposed. This formation of a final particle is free of indication for ordered or repetitive arrays and is therefore considered less structured compared to PC particles. The cryo-TEM images FIGS. 8(C-D) and FIG. 9(B) clearly shows the different surface composition for the different assembly routes. The sequential assembly of polymer and lipid as for PS_{HIGH} (FIG. 8(C)) and PC_{LOW} (FIG. 8(D)) leads to the formation of a different structure that causes different surface properties depending on the assembly protocol. In both cases, nanoparticles in the size range of 50 to 300 nm are observed. However, contrary to the very compact, vesicular like, clear shaped PS_{HIGH} particles, the PC_{LOW} particles have a highly lamellar structure, resulting in very different surface properties. The single-step assembly of MP_{MID} (FIG. 9(B) particles leads to heterogenous particles containing both attributes, vesicular-like and lamellar structures. They show an inner cavity and not very high density. The different particle systems can therefore be distinguished in the cryo-TEM images. The corresponding X-ray scattering curve (FIG. 9(A)) shows a small peak at approximately 0.1 Å⁻¹, confirming the partly lamellar areas that can be seen in the cryo-TEM picture (FIG. 9(B)). The X-ray scattering curve of the MP_{MID} intermediate in FIG. 9(A), consisting of two molecular species that repel each other (DOTAP and Protamine) shows no ordered, repetitive structures.

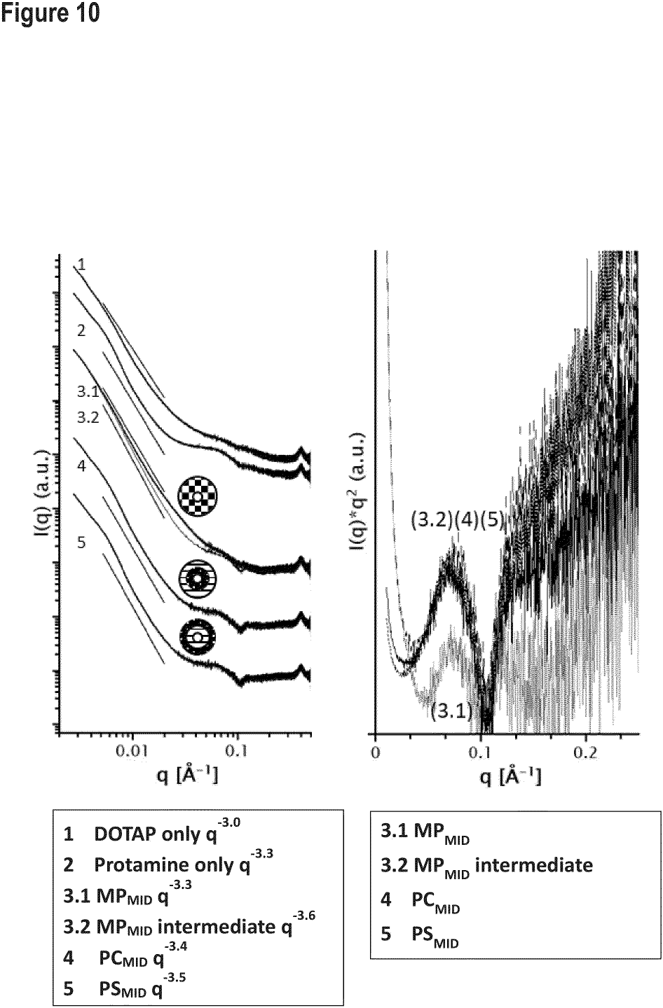

FIG. 10 shows the small angle x-ray scattering curves of pure DOTAP, protamine and the hybrid nanoparticles from the 3 different particle topologies with 45 % protamine as either shell (PS), core (PC) or self-assembled (MP) component. The scattering curves of DOTAP (curve number 1) and protamine (curve number 2) can be clearly distinguished from the other curves. The scattering curves of the final complex particles are in general similar. Only for the scattering of the triple complex MP_{MID} particle (curve number 3.1), a higher scattering is observed at q-values of 0.1 Å⁻¹ in comparison to PC_{MID} (curve number 4), PS_{MID} (curve number 5) and the MP_{MID} intermediate (curve number 3.2). SAXS scattering data gained of the PS_{high} and PC_{low} particles confirm the SANS data (not shown). Instead, a residual signal from background subtraction is observable in some scattering curves at ~0.1 Å⁻¹ which is not considered to be meaningful. For all particles a power-law scattering of, l(q) oc q^{-P} with 3<P<4 is observed. The superstructures formed from intermediate particles (MP_{MID} intermediate) tend to have a less rough surface and higher globularity (P ~ 3.6) compared to that of the final particle (P ~3.3). Same accounts for the intermediate PS_{MID} and PC_{MID} particles (data not shown). Between the different particle systems, the power law is trending. The self-assembled hybrid particles have the lowest P value, indicating a rather rough surface (MP_{MID}, 3.3). This indicates a more heterogeneous surface composition comprising of lipid, polymer and mRNA in comparison to the core/shell assembled PC/PS particles. A comparison of the Porod exponents between the SAXS and SANS measurements is not considered meaningful, as the buffer composition, as well as the experimental set up is not comparable.

Kratky plots of the scattering curves were determined by plotting I(q)*q² against q. A parabola like peak in the Kratky plot indicates the presence of folded domains. The Kratky-plot (right plot) shows a peak at 0.075 Å⁻¹ for all particles. However, this peak is more pronounced for PS_{MID}, PC_{MID} in comparison to MP_{MID}, indicating that we are dealing with compact particles with different topologies.

1. An RNA particle comprising:
(i) RNA,
(ii) at least one cationic or cationically ionizable lipid or lipid-like material, and
(iii) at least one cationic polymer,
wherein the particle does not have a core-shell structure.

2. The RNA particle of claim 1, which is obtainable by a process comprising the following steps:
a. obtaining a mixture comprising at least one cationic or cationically ionizable lipid or lipid-like material in an organic solvent;
b. removing the organic solvent from the mixture obtained in step a. thus obtaining a lipid film;
c. adding a solution comprising at least one cationic polymer to the lipid film obtained in step b.;
d. agitating the mixture obtained in step c. to obtain a colloid; and
e. mixing the colloid obtained in step d. with RNA to obtain RNA particles.

3. An RNA particle comprising:
(i) RNA,
(ii) at least one cationic or cationically ionizable lipid or lipid-like material, and
(iii) at least one cationic polymer,
wherein the RNA particle is obtainable by a process comprising the following steps:
a. obtaining a mixture comprising at least one cationic or cationically ionizable lipid or lipid-like material in an organic solvent;
b. removing the organic solvent from the mixture obtained in step a. thus obtaining a lipid film;
c. adding a solution comprising at least one cationic polymer to the lipid film obtained in step b.;
d. agitating the mixture obtained in step c. to obtain a colloid; and
e. mixing the colloid obtained in step d. with RNA to obtain RNA particles.

4. The RNA particle of claim 2 or 3, wherein step c. results in hydration of the lipid film.

5. The RNA particle of any one of claims 2 to 4, wherein step d. comprises reducing the size of the particles of the colloid obtained.

6. The RNA particle of any one of claims 2 to 5, wherein step d. comprises applying sonic energy or mechanical energy.

7. The RNA particle of any one of claims 2 to 6, wherein step d. comprises one or more selected from the group consisting of sonification, extrusion, dual asymmetric centrifugation, vortexing and compounding.

8. The RNA particle of any one of claims 2 to 7, wherein step d. comprises dual asymmetric centrifugation.

9. The RNA particle of any one of claims 2 to 8, wherein the process does not comprise preparing complexes by mixing RNA and cationic polymer in the absence of cationic or cationically ionizable lipid or lipid-like material.

10. The RNA particle of any one of claims 2 to 9, wherein the process does not comprise preparing a colloid comprising cationic or cationically ionizable lipid or lipid-like material in the absence of cationic polymer.

11. The RNA particle of any one of claims 2 to 10, wherein the process does not comprise mixing RNA with cationic polymer in the absence of cationic or cationically ionizable lipid or lipid-like material.

12. The RNA particle of any one of claims 2 to 11, wherein the process does not comprise mixing RNA with cationic or cationically ionizable lipid or lipid-like material in the absence of cationic polymer.

13. The RNA particle of any one of claims 1 to 12, wherein one or more, preferably all of the components (i), (ii) and (iii) are distributed throughout the particle.

14. The RNA particle of any one of claims 1 to 13, which does not consist of a particle core comprising RNA and an outer shell.

15. The RNA particle of claim 14, wherein the particle core comprises RNA and cationic polymer and the outer shell is a lipid shell comprising cationic or cationically ionizable lipid or lipid-like material.

16. The RNA particle of claim 14, wherein the particle core comprises RNA and cationic or cationically ionizable lipid or lipid-like material and the outer shell comprises cationic polymer.

17. The RNA particle of any one of claims 1 to 16, wherein the cationically ionizable lipid or lipid-like material is cationic only at acidic pH and does not remain cationic at neutral pH.

18. The RNA particle of any one of claims 1 to 17, wherein the cationic or cationically ionizable lipid or lipid-like material comprises N-(1-(2,3-dioleoyloxy)propyl)-N,N,N-trimethylammonium chloride (DOTAP), N,N-dimethyl-2,3-dioleyloxypropylamine (DODMA), N,N-dioleyl-N,N-dimethylammonium chloride (DODAC), N,N-distearyl-N,N-dimethylammonium bromide (DDAB), N-(1-(2,3-dioleyloxy)propyl)-N,N,N-trimethylammonium chloride (DOTMA), 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-KC2-DMA), 2,2-dilinoleyl-4-dimethylaminomethyl-[1,3]-dioxolane (DLin-K-DMA), 1,2-dimyristyloxypropyl-3-dimethyl-hydroxy ethyl ammonium bromide (DMRIE), (6Z,9Z,28Z,31Z)-heptatriaconta-6,9,28,31-tetraen-19-yl 4-(dimethylamino)butanoate (D-Lin-MC3-DMA) or a mixture thereof.

19. The RNA particle of any one of claims 1 to 18, wherein the cationic or cationically ionizable lipid or lipid-like material comprises N-(1-(2,3-dioleoyloxy)propyl)-N,N,N-trimethylammonium chloride (DOTAP).

20. The RNA particle of any one of claims 1 to 19, wherein the cationic polymer is a cationic peptide or protein.

21. The RNA particle of any one of claims 1 to 20, wherein the cationic polymer comprises one or more selected from the group consisting of protamine, spermidine (N-[3-aminopropyl]-1,4-butanediamine), DEAE-dextran, chitosan, polyethyleneimine and poly-lysine.

22. The RNA particle of any one of claims 1 to 21, wherein the cationic polymer comprises protamine.

23. The RNA particle of any one of claims 1 to 22, wherein the RNA is mRNA or saRNA.

24. The RNA particle of any one of claims 1 to 23, wherein the particle is a nanoparticle.

25. The RNA particle of any one of claims 1 to 24, wherein the particle has a size of from about 100 nm to about 300 nm.

26. The RNA particle of any one of claims 1 to 25, wherein the particle is a non-viral particle.

27. A composition comprising a plurality of the particles of any one of claims 1 to 26.

28. A method for delivering RNA to cells of a subject, the method comprising administering to a subject a plurality of the particles of any one of claims 1 to 26 or the composition of claim 27.

29. A method for delivering a therapeutic peptide or protein to a subject, the method comprising administering to a subject a plurality of the particles of any one of claims 1 to 26 or the composition of claim 27, wherein the RNA encodes the therapeutic peptide or protein.

30. A method for treating or preventing a disease or disorder in a subject, the method comprising administering to a subject a plurality of the particles of any one of claims 1 to 26 or the composition of claim 27, wherein delivering the RNA to cells of the subject is beneficial in treating or preventing the disease or disorder.

31. A method for treating or preventing a disease or disorder in a subject, the method comprising administering to a subject a plurality of the particles of any one of claims 1 to 26 or the composition of claim 27, wherein the RNA encodes a therapeutic peptide or protein and wherein delivering the therapeutic peptide or protein to the subject is beneficial in treating or preventing the disease or disorder.

32. The method of any one of claims 28 to 31, wherein the subject is a mammal.

33. The method of claim 32, wherein the mammal is a human.

34. A process for the preparation of RNA particles comprising the following steps:
a. obtaining a mixture comprising at least one cationic or cationically ionizable lipid or lipid-like material in an organic solvent;
b. removing the organic solvent from the mixture obtained in step a. thus obtaining a lipid film;
c. adding a solution comprising at least one cationic polymer to the lipid film obtained in step b.;
d. agitating the mixture obtained in step c. to obtain a colloid; and
e. mixing the colloid obtained in step d. with RNA to obtain RNA particles.

35. The process of claim 34, wherein step c. results in hydration of the lipid film.

36. The process of claim 34 or 35, wherein step d. comprises reducing the size of the particles of the colloid obtained.

37. The process of any one of claims 34 to 36, wherein step d. comprises applying sonic energy or mechanical energy.

38. The process of any one of claims 34 to 37, wherein step d. comprises one or more selected from the group consisting of sonification, extrusion, dual asymmetric centrifugation, vortexing and compounding.

39. The process of any one of claims 34 to 38, wherein step d. comprises dual asymmetric centrifugation.

40. The process of any one of claims 34 to 39, wherein the process does not comprise preparing complexes by mixing RNA and cationic polymer in the absence of cationic or cationically ionizable lipid or lipid-like material.

41. The process of any one of claims 34 to 40, wherein the process does not comprise preparing a colloid comprising cationic or cationically ionizable lipid or lipid-like material in the absence of cationic polymer.

42. The process of any one of claims 34 to 41, wherein the process does not comprise mixing RNA with cationic polymer in the absence of cationic or cationically ionizable lipid or lipid-like material.

43. The process of any one of claims 34 to 42, wherein the process does not comprise mixing RNA with cationic or cationically ionizable lipid or lipid-like material in the absence of cationic polymer.

44. The process of any one of claims 34 to 43, wherein the RNA particles are particles according to any one of claims 1 to 26.
